(19) United States
(12) Patent Application Publication
(10) Pub. No.: US 2013/0096135 A1
(43) Pub. Date: Apr. 18, 2013

(54) Selected Inhibitors of Protein Tyrosine Kinase Activity

(71) Applicant: MethylGene Inc., Montreal (CA)

(72) Inventors: Stéphane Raeppel, St. Lazare, Quebec (CA); Franck Raeppel, Montreal, Quebec (CA); Masashi Kishida, Osaka (JP); Seiji Hata, Osaka (JP); Yohei Yuki, Osaka (JP); Arkadii Vaisburg, Kirkland, Quebec (CA)

(73) Assignee: METHYLGENE INC., Montreal (CA)

(21) Appl. No.: 13/628,975

(22) Filed: Sep. 27, 2012

Related U.S. Application Data
(60) Provisional application No. 61/541,317, filed on Sep. 30, 2011.

(51) Int. Cl. A61K 31/496; A61K 31/4545; C07D 401/14

(52) U.S. Cl. CPC A61K 31/496; C07D 401/14; A61K 31/4545
USPC 514/253.04; 546/114; 514/301; 544/362

(57) ABSTRACT

The present invention provides new compounds and methods for treating a disease responsive to inhibition of kinase activity, for example a disease responsive to inhibition of protein tyrosine kinase activity, for example a disease responsive to inhibition of protein tyrosine kinase activity of growth factor receptors, for example a disease responsive to inhibition of receptor type tyrosine kinase signaling, or for example, a disease responsive to inhibition of VEGF receptor signaling.

### BACKGROUND OF THE INVENTION

1. Field of the Invention

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling.

2. Summary of the Related Art

Tyrosine kinases may be classified as growth factor receptor (e.g. EGFR, PDGFR, FGFR and erbB2) or non-receptor (e.g. c-src and bcr-abl) kinases. The receptor type tyrosine kinases make up about 20 different subfamilies. The non-receptor type tyrosine kinases make up numerous subfamilies. These tyrosine kinases have diverse biological activity. Receptor tyrosine kinases are large enzymes that span the cell membrane and possess an extracellular binding domain for growth factors, a transmembrane domain, and an intracellular portion that functions as a kinase to phosphorylate a specific tyrosine residue in proteins and hence to influence cell proliferation. Aberrant or inappropriate protein kinase activity can contribute to the rise of disease states associated with such aberrant kinase activity.

Angiogenesis is an important component of certain normal physiological processes such as embryogenesis and wound healing, but aberrant angiogenesis contributes to some pathological disorders and in particular to tumor growth. VEGF-A (vascular endothelial growth factor A) is a key factor promoting neovascularization (angiogenesis) of tumors. VEGF induces endothelial cell proliferation and migration by signaling through two high affinity receptors, the fins-like tyrosine kinase receptor, Flt-1, and the kinase insert domain-containing receptor, KDR. These signaling responses are critically dependent upon receptor dimerization and activation of intrinsic receptor tyrosine kinase (RTK) activity. The binding of VEGF as a disulfide-linked homodimer stimulates receptor dimerization and activation of the RTK domain. The kinase autophosphorylates cytoplasmic receptor tyrosine residues, which then serve as binding sites for molecules involved in the propagation of a signaling cascade. Although multiple pathways are likely to be elucidated for both receptors, KDR signaling is most extensively studied, with a mitogenic response suggested to involve ERK-1 and ERK-2 mitogen-activated protein kinases.

Disruption of VEGF receptor signaling is a highly attractive therapeutic target in cancer, as angiogenesis is a prerequisite for all solid tumor growth, and that the mature endothelium remains relatively quiescent (with the exception of the female reproductive system and wound healing). A number of experimental approaches to inhibiting VEGF signaling have been examined, including use of neutralizing antibodies, receptor antagonists, soluble receptors, antisense constructs and dominant-negative strategies.

Tyrosine kinases also contribute to the pathology of ophthalmic diseases, disorders and conditions, such as age-related macular degeneration (AMD) and diabetic retinopathy (DR). Blindness from such diseases has been linked to anomalies in retinal neovascularization. The formation of new blood vessels is regulated by growth factors such as VEGF and HGF that activate receptor tyrosine kinases resulting in the initiation of signaling pathways leading to plasma leakage into the macula, causing vision loss. Kinases are thus attractive targets for the treatment of eye diseases involving neovascularization.

Thus, there is a need to develop a strategy for controlling neovascularization of the eye and to develop a strategy for the treatment of ocular diseases.

Here we describe small molecules that are potent inhibitors of protein tyrosine kinase activity.

### BRIEF SUMMARY OF THE INVENTION

The present invention provides new compounds and methods for treating a disease responsive to inhibition of kinase activity, for example a disease responsive to inhibition of protein tyrosine kinase activity, for example a disease responsive to inhibition of protein tyrosine kinase activity of growth factor receptors, for example a disease responsive to inhibition of receptor type tyrosine kinase signaling, or for example, a disease responsive to inhibition of VEGF receptor signaling. In some embodiments the disease is a cell proliferative disease. In other embodiments, the disease is an ophthalmic disease. The compounds of the invention are inhibitors of kinase activity, such as protein tyrosine kinase activity, for example protein tyrosine kinase activity of growth factor receptors, or for example receptor type tyrosine kinase signaling.

In a first aspect, the invention provides compounds that are useful as kinase inhibitors and N-oxides, hydrates, solvates, tautomers, pharmaceutically acceptable salts, prodrugs, soft drugs and complexes thereof, and racemic and scalemic mixtures, diastereomers and enantiomers thereof. Because compounds of the present invention are useful as kinase inhibitors they are, therefore, useful research tools for the study of the role of kinases in both normal and disease states. In some embodiments, the invention provides compounds that are useful as inhibitors of VEGF receptor signaling and, therefore, are useful research tools for the study of the role of VEGF in both normal and disease states.

In a second aspect, the invention provides compositions comprising a compound according to the present invention and a pharmaceutically acceptable carrier, excipient or diluent. For example, the invention provides compositions comprising a compound that is an inhibitor of VEGF receptor signaling, or a pharmaceutically acceptable salt thereof, and a pharmaceutically acceptable carrier, excipient, or diluent.

In a third aspect, the invention provides a method of inhibiting kinase activity, for example protein tyrosine kinase, for example tyrosine kinase activity of a growth factor receptor, the method comprising contacting the kinase with a compound according to the present invention, or with a composition according to the present invention. In some embodiments of this aspect, the invention provides a method of inhibiting receptor type tyrosine kinase signaling, for example inhibiting VEGF receptor signaling Inhibition can be in a cell or a multicellular organism. If in a cell, the method according to this aspect of the invention comprises contacting the cell with a compound according to the present invention, or with a composition according to the present invention. If in a multicellular organism, the method according to this aspect of the invention comprises administering to the organism a compound according to the present invention, or a composition according to the present invention. In some embodiments the organism is a mammal, for example a primate, for example a human.

In a fourth aspect, the invention provides a method of inhibiting angiogenesis, the method comprising administering to a patient in need thereof a therapeutically effective amount of a compound according to the present invention, or a therapeutically effective amount of a composition according to the present invention. In some embodiments of this aspect, the angiogenesis to be inhibited is involved in tumor growth. In some other embodiments the angiogenesis to be inhibited is retinal angiogenesis. In some embodiments of this aspect, the patient is a mammal, for example a primate, for example a human.

In a fifth aspect, the invention provides a method of treating a disease responsive to inhibition of kinase activity, for example a disease responsive to inhibition of protein tyrosine kinase activity, for example a disease responsive to inhibition of protein tyrosine kinase activity of growth factor receptors. In some embodiments of this aspect, the invention provides a method of treating a disease responsive to inhibition of receptor type tyrosine kinase signaling, for example a disease responsive to inhibition of VEGF receptor signaling, the method comprising administering to an organism in need thereof a therapeutically effective amount of a compound according to the present invention, or a composition according to the present invention. In some embodiments of this aspect, the organism is a mammal, for example a primate, for example a human.

In a sixth aspect, the invention provides a method of treating a cell proliferative disease, the method comprising administering to a patient in need thereof a therapeutically effective amount of a compound according to the present invention, or a therapeutically effective amount of a composition according to the present invention. In some embodiments of this aspect, the cell proliferative disease is cancer. In some embodiments, the patient is a mammal, for example a primate, for example a human.

In a seventh aspect, the invention provides a method of treating an ophthalmic disease, disorder or condition, the method comprising administering to a patient in need thereof a therapeutically effective amount of a compound according to the present invention, or a therapeutically effective amount of a composition according to the present invention. In some embodiments of this aspect, the disease is caused by choroidal angiogenesis. In some embodiments of this aspect, the patient is a mammal, for example a primate, for example a human.

In an eighth aspect, the invention provides for the use of a compound according to the present invention for or in the manufacture of a medicament to inhibit kinase activity, for example to inhibit protein tyrosine kinase activity, for example to inhibit protein tyrosine kinase activity of growth factor receptors. In some embodiments of this aspect, the invention provides for the use of a compound according to the present invention for or in the manufacture of a medicament to inhibit receptor type tyrosine kinase signaling, for example to inhibit VEGF receptor signaling. In some embodiments of this aspect, the invention provides for the use of a compound according to the present invention for or in the manufacture of a medicament to treat a disease responsive to inhibition of kinase activity. In some embodiments of this aspect, the disease is responsive to inhibition of protein tyrosine kinase activity, for example inhibition of protein tyrosine kinase activity of growth factor receptors. In some embodiments of this aspect, the disease is responsive to inhibition of receptor type tyrosine kinase signaling, for example VEGF receptor signaling. In some embodiments of this aspect, the disease is a cell proliferative disease, for example cancer. In some embodiments of this aspect, the disease is an ophthalmic disease, disorder or condition. In some embodiments of this aspect, the ophthalmic disease, disorder or condition is caused by choroidal angiogenesis. In some embodiments of this aspect, the disease is age-related macular degeneration, diabetic retinopathy or retinal oedema.

In a ninth aspect, the invention provides for the use of a compound according to the present invention, or a composition thereof, to inhibit kinase activity, for example to inhibit receptor type tyrosine kinase activity, for example to inhibit protein tyrosine kinase activity of growth factor receptors. In some embodiments of this aspect, the invention provides for the use of a compound according to the present invention, or a composition thereof, to inhibit receptor type tyrosine kinase signaling, for example to inhibit VEGF receptor signaling.

In a tenth aspect, the invention provides for the use of a compound according to the present invention, or a composition thereof, to treat a disease responsive to inhibition of kinase activity, for example a disease responsive to inhibition of protein tyrosine kinase activity, for example a disease responsive to inhibition or protein tyrosine kinase activity of growth factor receptors. In some embodiments of this aspect, the invention provides for the use of a compound according to the present invention, or a composition thereof, to treat a disease responsive to inhibition of receptor type tyrosine kinase signaling, for example a disease responsive to inhibition of VEGF receptor signaling. In some embodiments of this aspect, the disease is a cell proliferative disease, for example cancer. In some embodiments of this aspect, the disease is an ophthalmic disease, disorder or condition. In some embodiments of this aspect, the ophthalmic disease, disorder or condition is caused by choroidal angiogenesis.

The foregoing merely summarizes some aspects of the invention and is not intended to be limiting in nature. These aspects and other aspects and embodiments are described more fully below.

### DETAILED DESCRIPTION

The invention provides compounds, compositions and methods for inhibiting kinase activity, for example protein tyrosine kinase activity, for example receptor protein kinase activity, for example the VEGF receptor KDR. The invention also provides compounds, compositions and methods for inhibiting angiogenesis, treating a disease responsive to inhibition of kinase activity, treating cell proliferative diseases and conditions and treating ophthalmic diseases, disorders and conditions. The patent and scientific literature referred to herein reflects knowledge that is available to those with skill in the art. The issued patents, published patent applications, and references that are cited herein are hereby incorporated by reference to the same extent as if each was specifically and individually indicated to be incorporated by reference. In the case of inconsistencies, the present disclosure will prevail.

For purposes of the present invention, the following abbreviations will be used (unless expressly stated otherwise)

| | |
|---|---|
| Ac | acetyl |
| AcOEt | ethyl acetate |
| AcOH | acetic acid |
| aq | aqueous |
| bd | broad doublet (NMR) |
| Bn | benzyl |
| Boc | tert-butoxycarbonyl |
| br s | broad singlet (NMR) |
| CV | column volume |
| d | doublet (NMR) |
| dd | doublet of doublets (NMR) |
| DCC | dicyclohexyl carbodiimide |
| DCM | dichloromethane |
| DEAD | diethyl diazenedicarboxylate |
| DIPEA | diisopropyl ethylamine |
| DMAP | N,N-dimethylamino pyridine |
| DMF | N,N-dimethylformamide |
| DMSO | dimethylsulfoxide |
| DMSO-d₆ | dimethylsulfoxide-d₆ |
| EDC | 1-(3-dimethylaminopropyl)-3-ethyl-carbodiimide |
| Et | ethyl |
| EDCI | 1-(3-dimethylaminopropyl)-3-ethyl-carbodiimide |
| Et₃N | triethylamine |
| EtOH | ethanol |
| EtOAc | ethyl acetate |
| Et₂O | diethyl ether |
| equiv | equivalent |
| g | gram (grams) |
| h | hour (hours) |
| HOBT | 1-hydroxybenzotriazole |
| m | multiplet (NMR) |
| mL | milliliter |
| μL | microliter |
| Me | methyl |
| MeOH | methanol |
| MeOH-d₄ | methanol-d₄ |
| mg | milligram (milligrams) |
| min | minute (minutes) |
| MS | mass-spectroscopy |
| m/z | mass-to-charge ratio |
| NMP | N-methyl-2-pyrrolidone |
| NMR | nuclear magnetic resonance spectroscopy |
| PEG | polyethylene glycol |
| Ph | phenyl |
| Ppm | parts per million (NMR) |
| rt | room temperature |
| s | singlet (NMR) |
| t | triplet (NMR) |
| TFA | trifluoroacetic acid |
| THF | tetrahydrofuran |

For purposes of the present invention, the following definitions will be used (unless expressly stated otherwise):

The terms “kinase inhibitor” and “inhibitor of kinase activity”, and the like, are used to identify a compound which is capable of interacting with a kinase and inhibiting its enzymatic activity.

The term “inhibiting kinase enzymatic activity” and the like is used to mean reducing the ability of a kinase to transfer a phosphate group from a donor molecule, such as adenosine tri-phosphate (ATP), to a specific target molecule (substrate). For example, the inhibition of kinase activity may be at least about 10%. In some embodiments of the invention, such reduction of kinase activity is at least about 25%, alternatively at least about 50%, alternatively at least about 75%, and alternatively at least about 90%. In other embodiments, kinase activity is reduced by at least 95% and alternatively by at least 99%. The IC₅₀ value is the concentration of kinase inhibitor which reduces the activity of a kinase to 50% of the uninhibited enzyme.

The terms “inhibitor of VEGF receptor signaling” is used to identify a compound having a structure as defined herein, which is capable of interacting with a VEGF receptor and inhibiting the activity of the VEGF receptor. In some embodiments, such reduction of activity is at least about 50%, alternatively at least about 75%, and alternatively at least about 90%. In some embodiments, activity is reduced by at least 95% and alternatively by at least 99%.

The term “inhibiting effective amount” is meant to denote a dosage sufficient to cause inhibition of kinase activity. The amount of a compound of the invention which constitutes an “inhibiting effective amount” will vary depending on the compound, the kinase, and the like. The inhibiting effective amount can be determined routinely by one of ordinary skill in the art. The kinase may be in a cell, which in turn may be in a multicellular organism. The multicellular organism may be, for example, a plant, a fungus or an animal, for example a mammal and for example a human. The fungus may be infecting a plant or a mammal, for example a human, and could therefore be located in and/or on the plant or mammal.

In an exemplary embodiment, such inhibition is specific, i.e., the kinase inhibitor reduces the ability of a kinase to transfer a phosphate group from a donor molecule, such as ATP, to a specific target molecule (substrate) at a concentration that is lower than the concentration of the inhibitor that is required to produce another, unrelated biological effect. For example, the concentration of the inhibitor required for kinase inhibitory activity is at least 2-fold lower, alternatively at least 5-fold lower, alternatively at least 10-fold lower, and alternatively at least 20-fold lower than the concentration required to produce an unrelated biological effect.

Thus, the invention provides a method for inhibiting kinase enzymatic activity, comprising contacting the kinase with an inhibiting effective amount of a compound or composition according to the invention. In some embodiments, the kinase is in an organism. Thus, the invention provides a method for inhibiting kinase enzymatic activity in an organism, comprising administering to the organism an inhibiting effective amount of a compound or composition according to the invention. In some embodiments, the organism is a mammal, for example a domesticated mammal. In some embodiments, the organism is a human.

The term “therapeutically effective amount” as employed herein is an amount of a compound of the invention, that when administered to a patient, elicits the desired therapeutic effect. The therapeutic effect is dependent upon the disease being treated and the results desired. As such, the therapeutic effect can be treatment of a disease-state. Further, the therapeutic effect can be inhibition of kinase activity. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

In some embodiments, the therapeutic effect is inhibition of angiogenesis. The phrase “inhibition of angiogenesis” is used to denote an ability of a compound according to the present invention to retard the growth of blood vessels, such as blood vessels contacted with the inhibitor as compared to blood vessels not contacted. In some embodiments, angiogenesis is tumor angiogenesis. The phrase “tumor angiogenesis” is intended to mean the proliferation of blood vessels that penetrate into or otherwise contact a cancerous growth, such as a tumor. In some embodiments, angiogenesis is abnormal blood vessel formation in the eye.

In an exemplary embodiment, angiogenesis is retarded by at least 25% as compared to angiogenesis of non-contacted blood vessels, alternatively at least 50%, alternatively at least 75%, alternatively at least 90%, alternatively at least 95%, and alternatively, at least 99%. Alternatively, angiogenesis is inhibited by 100% (i.e., the blood vessels do not increase in size or number). In some embodiments, the phrase “inhibition of angiogenesis” includes regression in the number or size of blood vessels, as compared to non-contacted blood vessels. Thus, a compound according to the invention that inhibits angiogenesis may induce blood vessel growth retardation, blood vessel growth arrest, or induce regression of blood vessel growth.

Thus, the invention provides a method for inhibiting angiogenesis in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is treatment of an ophthalmic disease, disorder or condition. The phrase “treatment of an ophthalmic disease, disorder or condition” is intended to mean the ability of a compound according to the present invention to treat (a) a disease disorder or condition caused by choroidal angiogenesis, including, without limitation, age-related macular degeneration, or (b) diabetic retinopathy or retinal oedema. In some embodiments the phrase “treatment of an ophthalmic disease, disorder or condition” is intended to mean the ability of a compound according to the present invention to treat an exudative and/or inflammatory ophthalmic disease, disorder or condition, a disorder related to impaired retinal vessel permeability and/or integrity, a disorder related to retinal microvessel rupture leading to focal hemorrhage, a disease of the back of the eye, a retinal disease, or a disease of the front of the eye, or other ophthalmic disease, disorder or condition.

In some embodiments, the ophthalmic disease, disorder or condition includes but is not limited to Age Related Macular Degeneration (ARMD), exudative macular degeneration (also known as “wet” or neovascular age-related macular degeneration (wet-AMD), macular oedema, aged disciform macular degeneration, cystoid macular oedema, palpebral oedema, retinal oedema, diabetic retinopathy, Acute Macular Neuroretinopathy, Central Serous Chorioretinopathy, chorioretinopathy, Choroidal Neovascularization, neovascular maculopathy, neovascular glaucoma, obstructive arterial and venous retinopathies (e.g. Retinal Venous Occlusion or Retinal Arterial Occlusion), Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease(CAD), Frosted Branch Angitis, Sickle Cell Retinopathy and other Hemoglobinopathies, Angioid Streaks, macular oedema occurring as a result of aetiologies such as disease (e.g. Diabetic Macular Oedema), eye injury or eye surgery, retinal ischemia or degeneration produced for example by injury, trauma or tumours, uveitis, iritis, retinal vasculitis, endophthalmitis, panophthalmitis, metastatic ophthalmia, choroiditis, retinal pigment epithelitis, conjunctivitis, cyclitis, scleritis, episcleritis, optic neuritis, retrobulbar optic neuritis, keratitis, blepharitis, exudative retinal detachment, corneal ulcer, conjunctival ulcer, chronic nummular keratitis, Thygeson keratitis, progressive Mooren's ulcer, an ocular inflammatory disease caused by bacterial or viral infection or by an ophthalmic operation, an ocular inflammatory disease caused by a physical injury to the eye, and a symptom caused by an ocular inflammatory disease including itching, flare, oedema and ulcer, erythema, erythema exsudativum multiforme, erythema nodosum, erythema annulare, scleroedema, dermatitis, angioneurotic oedema, laryngeal oedema, glottic oedema, subglottic laryngitis, bronchitis, rhinitis, pharyngitis, sinusitis, laryngitis or otitis media.

In some embodiments, the ophthalmic disease, disorder or condition is (a) a disease disorder or condition caused by choroidal angiogenesis, including, without limitation, age-related macular degeneration, or (b) diabetic retinopathy or retinal oedema.

In some embodiments, the ophthalmic disease, disorder or condition includes but is not limited to age-related macular degeneration, diabetic retinopathy, retinal oedema, retinal vein occlusion, neovascular glaucoma, retinopathy of prematurity, pigmentary retinal degeneration, uveitis, corneal neovascularization or proliferative vitreoretinopathy.

In some embodiments, the ophthalmic disease, disorder or condition is age-related macular degeneration, diabetic retinopathy or retinal oedema.

Thus, the invention provides a method for treating an ophthalmic disease, disorder or condition in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is inhibition of retinal neovascularization. The phrase “inhibition of retinal neovascularization” is intended to mean the ability of a compound according to the present invention to retard the growth of blood vessels in the eye, for example new blood vessels originating from retinal veins, for example, to

In an exemplary embodiment, retinal neovascularization is retarded by at least 25% as compared to retinal neovascularization of non-contacted blood vessels, alternatively at least 50%, alternatively at least 75%, alternatively at least 90%, alternatively at least 95%, and alternatively, at least 99%. Alternatively, retinal neovascularization is inhibited by 100% (i.e., the blood vessels do not increase in size or number). In some embodiments, the phrase “inhibition of retinal neovascularization” includes regression in the number or size of blood vessels, as compared to non-contacted blood vessels. Thus, a compound according to the invention that inhibits retinal neovascularization may induce blood vessel growth retardation, blood vessel growth arrest, or induce regression of blood vessel growth.

Thus, the invention provides a method for inhibiting retinal neovascularization in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is inhibition of cell proliferation. The phrase “inhibition of cell proliferation” is used to denote an ability of a compound according to the present invention to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers or comparing the size of the growth of contacted cells with non-contacted cells.

In an exemplary embodiment, growth of cells contacted with the inhibitor is retarded by at least 25% as compared to growth of non-contacted cells, alternatively at least 50%, alternatively at least 75%, alternatively at least 90%, alternatively at least 95%, and alternatively, at least 99%. Alternatively, cell proliferation is inhibited by 100% (i.e., the contacted cells do not increase in number). In some embodiments, the phrase “inhibition of cell proliferation” includes a reduction in the number or size of contacted cells, as compared to non-contacted cells. Thus, a compound according to the invention that inhibits cell proliferation in a contacted cell may induce the contacted cell to undergo growth retardation, to undergo growth arrest, to undergo programmed cell death (i.e., to apoptose), or to undergo necrotic cell death.

In some embodiments, the contacted cell is a neoplastic cell. The term “neoplastic cell” is used to denote a cell that shows aberrant cell growth. In some embodiments, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some embodiments, the contacted cell is in an animal. Thus, the invention provides a method for treating a cell proliferative disease or condition in an animal, comprising administering to an animal in need of such treatment a therapeutically effective amount of a compound or composition of the invention. In some embodiments, the animal is a mammal, for example a domesticated mammal. In some embodiments, the animal is a human.

The term “cell proliferative disease or condition” is meant to refer to any condition characterized by aberrant cell growth, such as abnormally increased cellular proliferation. Examples of such cell proliferative diseases or conditions amenable to inhibition and treatment include, but are not limited to, cancer. Examples of particular types of cancer include, but are not limited to, breast cancer, lung cancer, colon cancer, rectal cancer, bladder cancer, prostate cancer, leukemia and renal cancer. In some embodiments, the invention provides a method for inhibiting neoplastic cell proliferation in an animal comprising administering to an animal having at least one neoplastic cell present in its body a therapeutically effective amount of a compound of the invention or a composition thereof

The term “patient” as employed herein for the purposes of the present invention includes humans and other animals, for example mammals, and other organisms. Thus the compounds, compositions and methods of the present invention are applicable to both human therapy and veterinary applications. In some embodiments the patient is a mammal, for example a human.

The terms “treating”, “treatment”, or the like, as used herein cover the treatment of a disease-state in an organism, and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, such as eliminating or curing of the disease. In some embodiments of the present invention the organism is an animal, for example a mammal, for example a primate, for example a human. As is known in the art, adjustments for systemic versus localized delivery, age, body weight, general health, sex, diet, time of administration, drug interaction, the severity of the condition, etc., may be necessary, and will be ascertainable with routine experimentation by one of ordinary skill in the art. In some embodiments, the terms “treating”, “treatment”, or the like, as used herein cover the treatment of a disease-state in an organism and includes at least one of (ii), (iii) and (iv) above.

Administration for non-ophthalmic diseases, disorders or conditions may be by any route, including, without limitation, parenteral, oral, sublingual, transdermal, topical, intranasal, intratracheal, or intrarectal. In some embodiments, compounds of the invention are administered intravenously in a hospital setting. In some embodiments, administration may be by the oral route.

Examples of routes of administration for ophthalmic diseases, disorders and conditions include but are not limited to, systemic, periocular, retrobulbar, intracanalicular, intravitral injection, topical (for example, eye drops), subconjunctival injection, subtenon, transcleral, intracameral, subretinal, electroporation, and sustained-release implant. Other routes of administration, other injection sites or other forms of administration for ophthalmic situations will be known or contemplated by one skilled in the art and are intended to be within the scope of the present invention.

In some embodiments of the present invention, routes of administration for ophthalmic diseases, disorders and conditions include topical, subconjunctival injection, intravitreal injection, or other ocular routes, systemically, or other methods known to one skilled in the art to a patient following ocular surgery.

In some other embodiments of the present invention, routes of administration for ophthalmic diseases, disorders and conditions include topical, intravitreal, transcleral, periocular, conjunctival, subtenon, intracameral, subretinal, subconjunctival, retrobulbar, or intracanalicular.

In some embodiments of the present invention, routes of administration for ophthalmic diseases, disorders and conditions include topical administration (for example, eye drops), systemic administration (for example, oral or intravenous), subconjunctival injection, periocular injection, intravitreal injection, and surgical implant for local delivery.

In some embodiments of the present invention, routes of administration for ophthalmic diseases, disorders and conditions include intravitreal injection, periocular injection, and sustained-release implant for local delivery.

In some embodiments of the present invention, an intraocular injection may be into the vitreous (intravitreal), under the conjunctiva (subconjunctival), behind the eye (retrobulbar), into the sclera, under the Capsule of Tenon (sub-Tenon), or may be in a depot form.

In some embodiments of the present invention, administration is local, including without limitation, topical, intravitreal, periorbital, intraocular, and other local administration to the eye, the ocular and/or periocular tissues and spaces, including without limitation, via a delivery device.

The compounds of the present invention form salts which are also within the scope of this invention.

The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic (exhibiting minimal or no undesired toxicological effects), physiologically acceptable) salts are preferred, although other salts are also useful, e.g., in isolation or purification steps which may be employed during preparation. Salts of the compounds of the invention may be formed, for example, by reacting a compound of the present invention with an amount of acid or base, such as an equivalent amount, in a medium such as one in which the salts precipitates or in an aqueous medium followed by lyophilization.

The compounds of the present invention which contain a basic moiety, such as but not limited to an amine or a pyridine or imidazole ring, may form salts with a variety of organic and inorganic acids. Examples of acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfanotes (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a carboxylic acid, may form salts with a variety of organic and inorganic bases. Examples of basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl and butyl chlorides, bromides and iodides), dialkyl sulfates (e.g. dimethyl, diethyl, dibuty and diamyl sulfates), long chain halides (e.g. decyl, lauryl, myristyl and stearyl chlorides, bromides and iodides), aralkyl halides (e.g. benzyl and phenethyl bromides), and others.

As used herein, the term “pharmaceutically acceptable salts” is intended to mean salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. Other salts include pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Another aspect of the invention provides compositions comprising a compound according to the present invention. For example, in some embodiments of the invention, a composition comprises a compound, or an N-oxide, hydrate, solvate, pharmaceutically acceptable salt, complex or prodrug,or soft drug of a compound according to the present invention present in at least about 30% enantiomeric or diastereomeric excess. In some embodiments of the invention, the compound, N-oxide, hydrate, solvate, pharmaceutically acceptable salt, complex or prodrug, or soft drug is present in at least about 50%, at least about 80%, or even at least about 90% enantiomeric or diastereomeric excess. In some embodiments of the invention, the compound, N-oxide, hydrate, solvate, pharmaceutically acceptable salt, complex or prodrug, or soft drug is present in at least about 95%, alternatively at least about 98% and alternatively at least about 99% enantiomeric or diastereomeric excess. In other embodiments of the invention, a compound, N-oxide, hydrate, solvate, pharmaceutically acceptable salt, complex or prodrug, or soft drug is present as a substantially racemic mixture.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of Formula (I) in at least about 30% diastereomeric or enantiomeric excess. In some embodiments of the invention, the compound is present in at least about 50% enantiomeric or diastereomeric excess, in at least about 80% enantiomeric or diastereomeric excess, or even in at least about 90% enantiomeric or diastereomeric excess. In some embodiments of the invention, the compound is present in at least about 95%, alternatively in at least about 98% enantiomeric or diastereomeric excess, and alternatively in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

The present invention also includes prodrugs of compounds of the invention. The term “prodrug” is intended to represent a compound covalently bonded to a carrier, which prodrug is capable of releasing the active ingredient when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the invention include compounds wherein a hydroxy, amino, carboxylic, or a similar group is modified. Examples of prodrugs include, but are not limited to esters (e.g., acetate, formate, phosphate and benzoate derivatives), carbamates (e.g., N,N-dimethylaminocarbonyl) of hydroxy or amino functional groups in compounds of the present invention), amides (e.g., trifluoroacetylamino, acetylamino, and the like), and the like.

The compounds of the invention may be administered, for example, as is or as a prodrug, for example in the form of an in vivo hydrolyzable ester or in vivo hydrolyzable amide. An in vivo hydrolyzable ester of a compound of the invention containing a carboxy or hydroxy group is, for example, a pharmaceutically acceptable ester which is hydrolyzed in the human or animal body to produce the parent acid or alcohol. Suitable pharmaceutically acceptable esters for carboxy include C₁-C₆alkoxymethyI esters (e.g., methoxymethyl), C₁-C₆alkanoyloxymethyl esters (e.g., for example pivaloyloxymethyl), phthalidyl esters, C₃-C₈cycloalkoxycarbonyloxy-C₁-C₆alkyl esters (e.g., 1-cyclohexylcarbonyloxyethyl); 1,3-dioxolen-2-onylmethyl esters (e.g., 5-methyl-1,3-dioxolen-2-onylmethyl; and C₁-C₆alkoxycarbonyloxyethyl esters (e.g., 1-methoxycarbonyloxyethyl) and may be formed at any appropriate carboxy group in the compounds of this invention.

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N-C₁-C₆alkyl or N,N-di-C₁-C₆alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention.

The compounds of the invention may be administered, for example, as is, as a prodrug or as a soft drug. How to make and administer prodrugs or soft drugs of the compounds of the invention is known to one skilled in the art

The present invention is also directed to solvates and hydrates of the compounds of the present invention. The term “solvate” refers to a molecular complex of a compound with one or more solvent molecules in a stoichiometric or non-stoichiometric amount. A molecular complex of a compound or moiety of a compound and a solvent can be stabilized by non-covalent intra-molecular forces such as, for example, electrostatic forces, van der Waals forces, or hydrogen bonds. Those skilled in the art of organic chemistry will appreciate that many organic compounds can form such complexes with solvents in which they are obtained, prepared or synthesized, or from which they are precipitated or crystallized. The term “hydrate” refers to a complex in which the one or more solvent molecules are water and includes monohydrates, hemi-hydrates, dihydrates, hexahydrates, and the like. The meaning of the words “solvate” and “hydrate” are well known to those skilled in the art. Techniques for the preparation of solvates are well established in the art (see, for example, Brittain, Polymorphism in Pharmaceutical solids. Marcel Dekker, New York, 1999; Hilfiker, Polymorphism in the Pharmaceutical Industry, Wiley, Weinheim, Germany, 2006).

In some embodiments of this aspect, the solvent is an inorganic solvent (for example, water). In some embodiments of this aspect, the solvent is an organic solvent (such as, but not limited to, alcohols, such as, without limitation, methanol, ethanol, isopropanol, and the like, acetic acid, ketones, esters, and the like). In certain embodiments, the solvent is one commonly used in the pharmaceutical art, is known to be innocuous to a recipient to which such solvate is administered (for example, water, ethanol, and the like) and in preferred embodiments, does not interfere with the biological activity of the solute.

The invention provides compounds that are useful as kinase inhibitors and N-oxides, hydrates, solvates, tautomers, pharmaceutically acceptable salts, prodrugs, soft drugs and complexes thereof, and racemic and scalemic mixtures, diastereomers and enantiomers thereof.

In some embodiments of the first aspect, the compounds are selected from the group consisting of

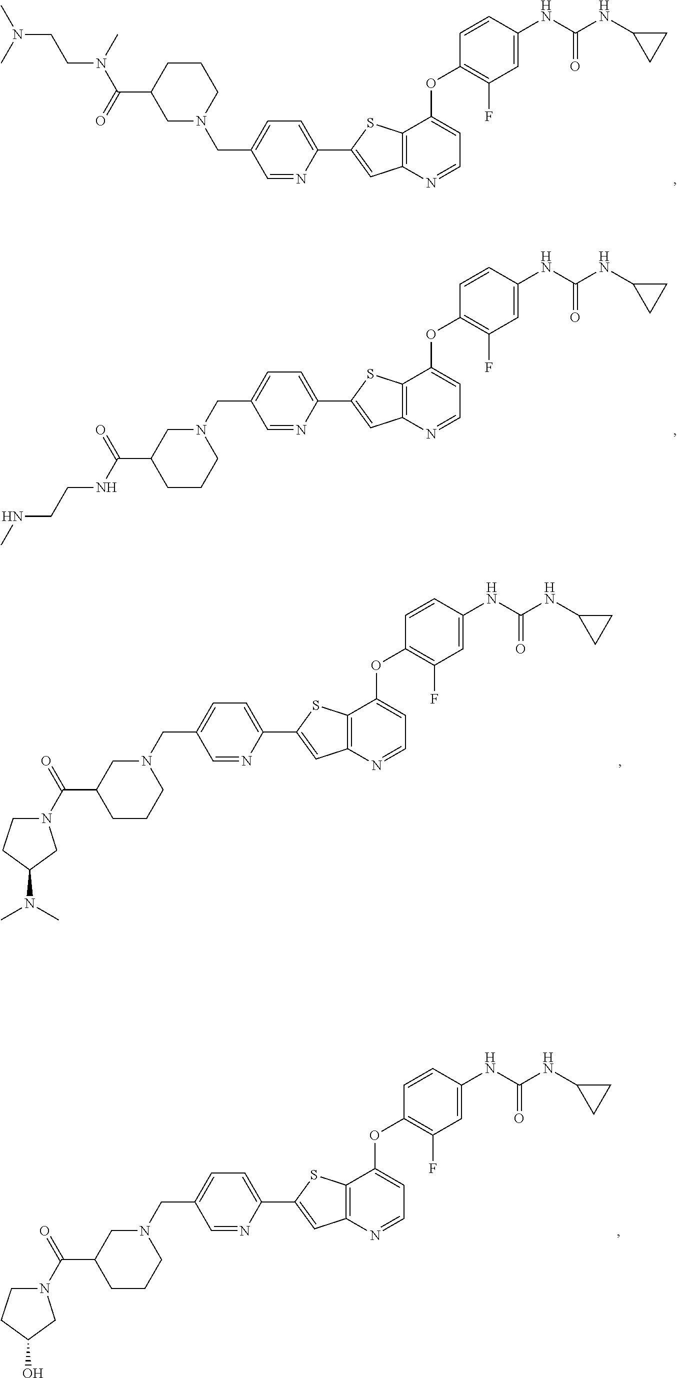

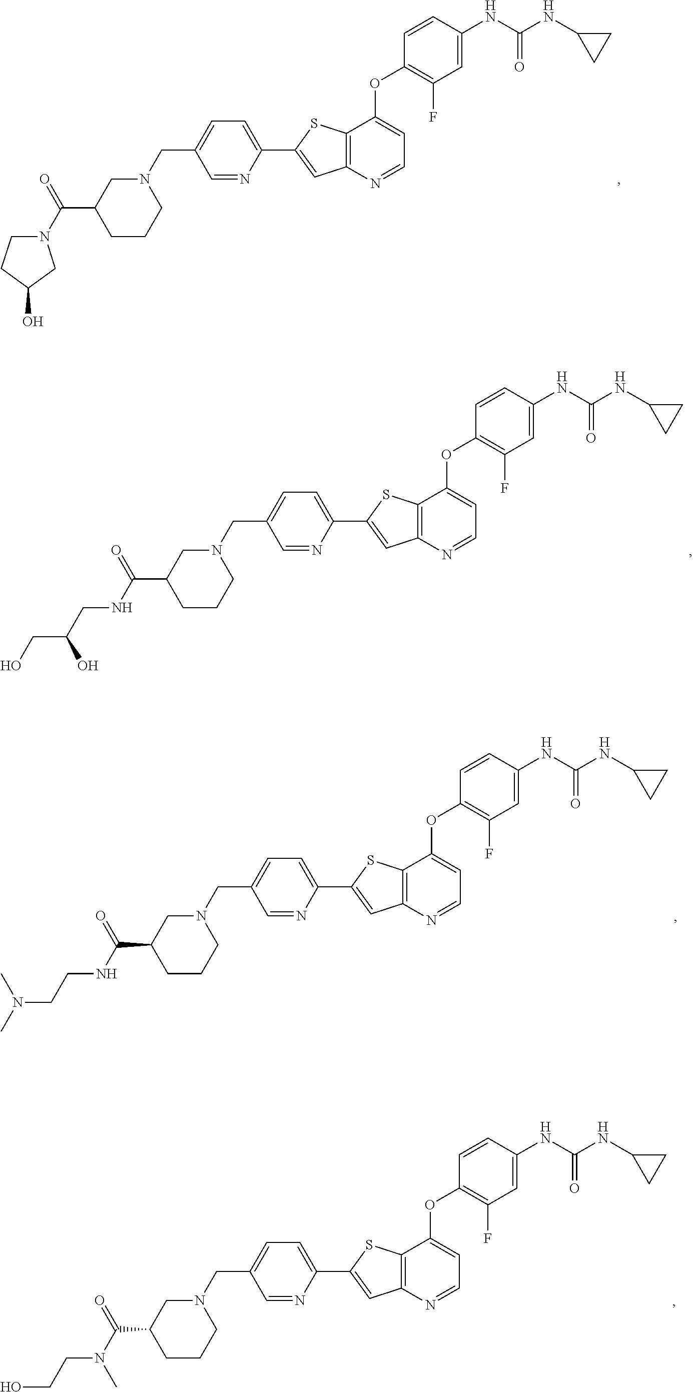

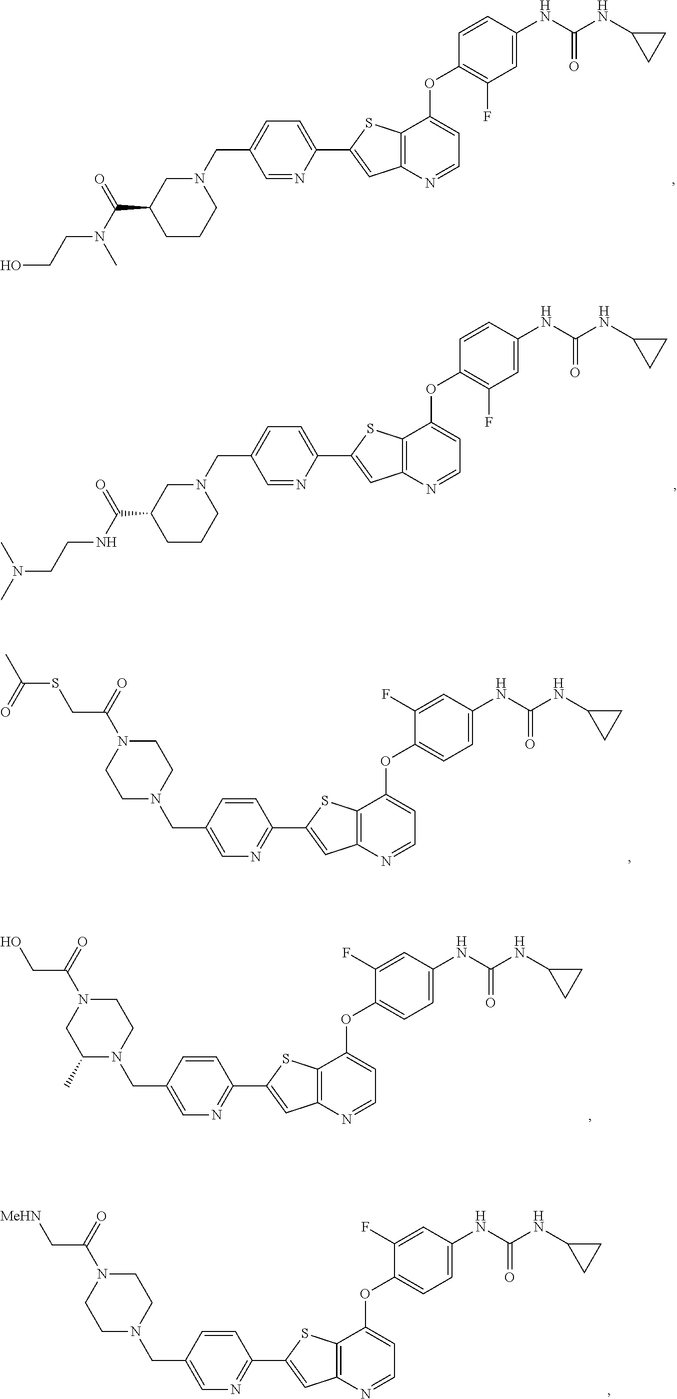

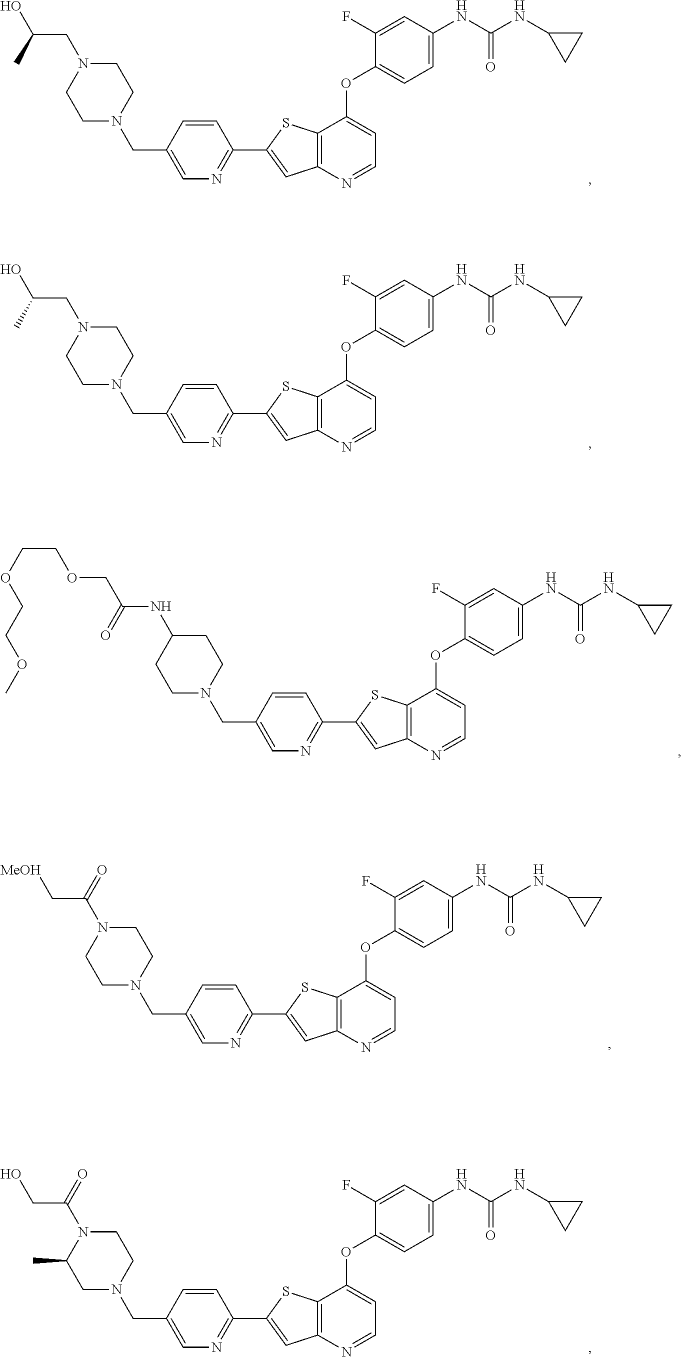

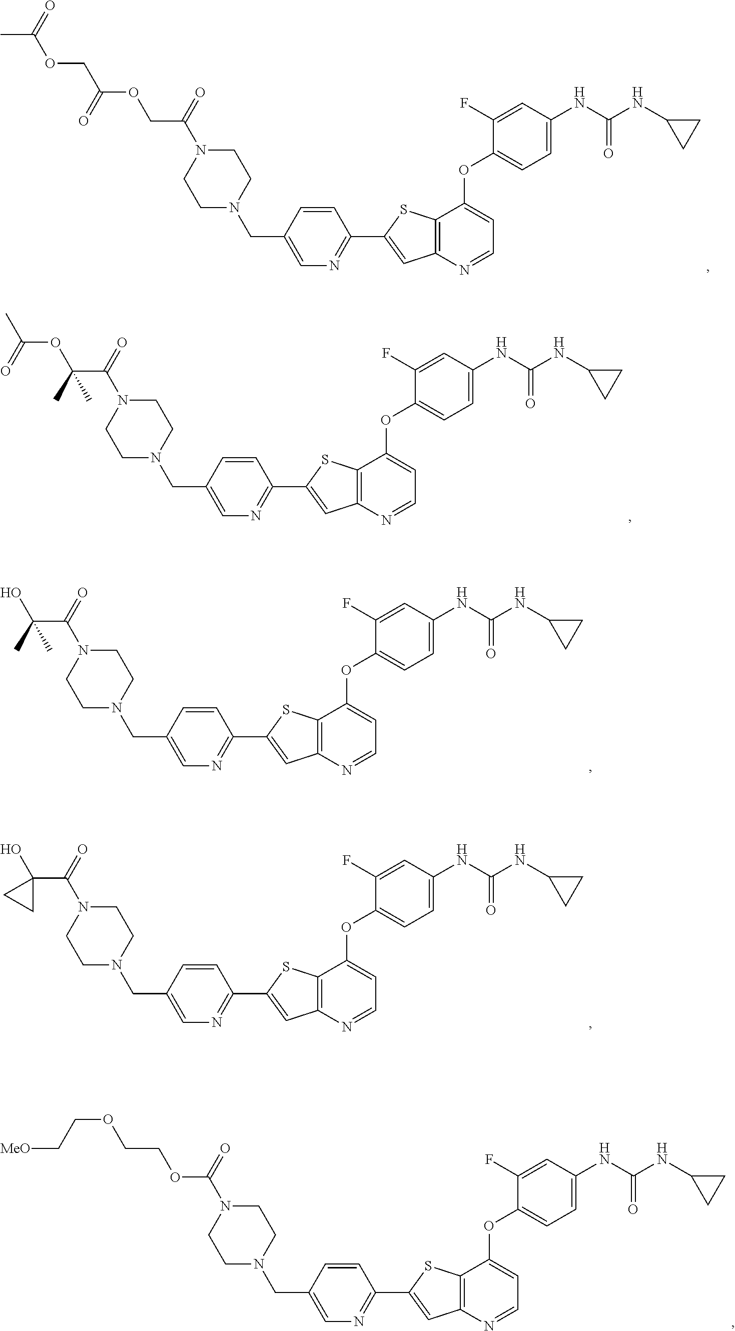

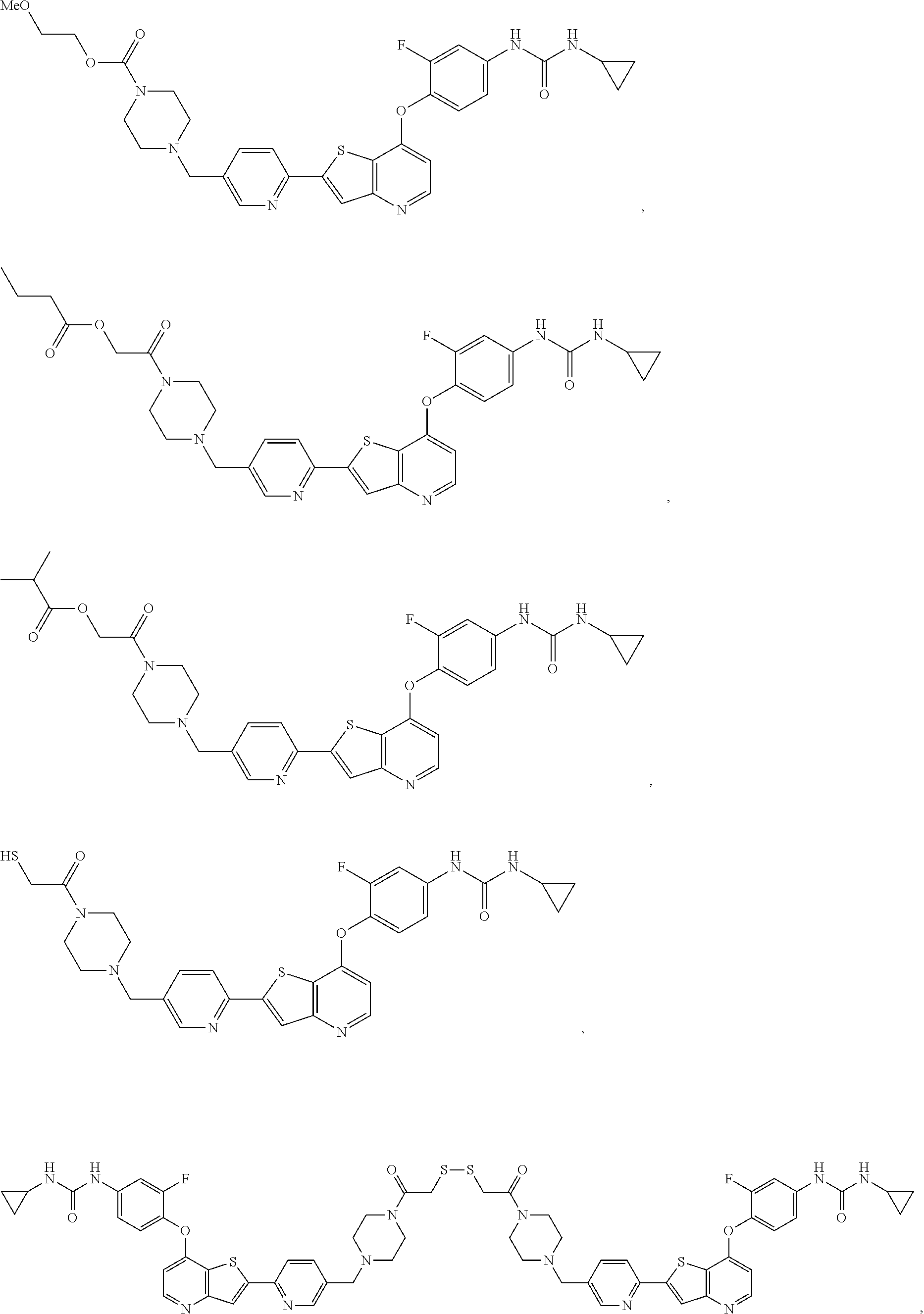

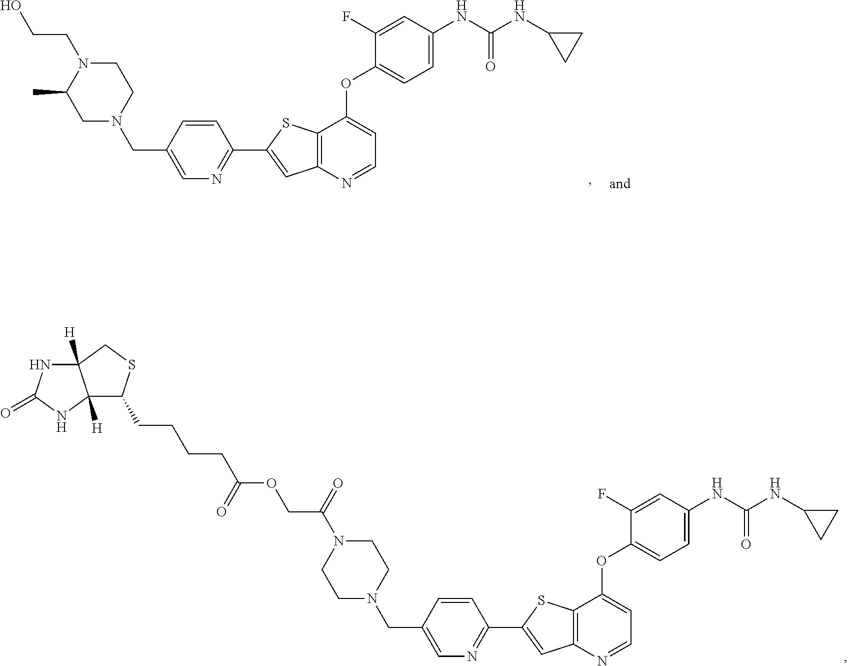 [SMILES: C[C@@H]1CN(Cc2ccc(-c3cc4nccc(Oc5ccc(NC(=O)NC6CC6)cc5F)c4s3)nc2)CCN1CCO ; "," ; "and" ; O=C(Nc1ccc(Oc2ccnc3cc(-c4ccc(CN5CCN(C(=O)COC(=O)CCCC[C@H]6SC[C@H]7NC(=O)N[C@H]76)CC5)cn4)sc23)c(F)c1)NC1CC1 ; ","]

including N-oxides, hydrates, solvates, tautomers, pharmaceutically acceptable salts, prodrugs, soft drugs and complexes thereof, and racemic and scalemic mixtures, diastereomers and enantiomers thereof.

Compounds of above formulas may generally be prepared according to the following Schemes. Tautomers and solvates (e.g., hydrates) of the compounds of above formulas are also within the scope of the present invention. Methods of solvation are generally known in the art. Accordingly, the compounds of the present invention may be in the free, hydrate or salt form, and may be obtained by methods exemplified by the following schemes below.

The following examples and preparations describe the manner and process of making and using the invention and are illustrative rather than limiting. It should be understood that there may be other embodiments which fall within the spirit and scope of the invention as defined by the claims appended hereto.

Compounds according to the invention include but are not limited to those described in the examples below. Compounds were named using Chemdraw Ultra (versions 10.0, 10.0.4 or version 8.0.3), which are available through Cambridgesoft (www.Cambridgesoft.com, 100 Cambridge Park Drive, Cambridge, Mass. 02140, or were derived therefrom.

The data presented herein demonstrate the inhibitory effects of the kinase inhibitors of the invention. These data lead one to reasonably expect that the compounds of the invention are useful not only for inhibition of kinase activity, protein tyrosine kinase activity, or embodiments thereof, such as, VEGF receptor signaling, but also as therapeutic agents for the treatment of proliferative diseases, including cancer and tumor growth and ophthalmic diseases, disorders and conditions.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

All reagents and solvents were obtained from commercial sources and used as received. ¹H-NMR spectra were recorded on a Mercury Plus Varian 400 MHz instrument in the solvents indicated. Low resolution mass-spectra (LRMS) were acquired on an Agilent MSD instrument. Analytical HPLC was performed on an Agilent 1100 instrument using Zorbax 3 μm, XDB-C8, 2.1×50 mm column; eluting with methanol/water containing 0.1% formic acid, with a gradient 5-95% methanol in 15 minutes. Automated column chromatography was performed on a Biotage SP1 or Biotage SP4 instruments using Biotage® SNAP, SiliaSep™ or SiliaFlash® cartridges. Flash column chromatography was performed using silica gel (cartriges SiliaFlash F60, 40-63 μM, pore size 60 Å, SiliCycle®).

Alternatively ¹H-NMR spectra were recorded on a JEOL AL300 300 MHz instrument in the solvents indicated. Low resolution mass-spectra (LRMS) were acquired on an Applied Biosystems/MDS Sciex 4000 QTRAP® instrument. Analytical HPLC was performed on a Shimazu SLC-10Avp machine; column Cadenza 5CD-C18, eluent water containing 0.1% TFA with a gradient of 5-95% MeCN over 15 minutes. Automated column chromatography was performed on a Yamazen Parallel Frac FR-260 apparatus (cartridges HI-FLASHTM COLUMN packed either with silicagel 40 μM or amino silicagel 40 μM)

### PARTICULAR EXAMPLES

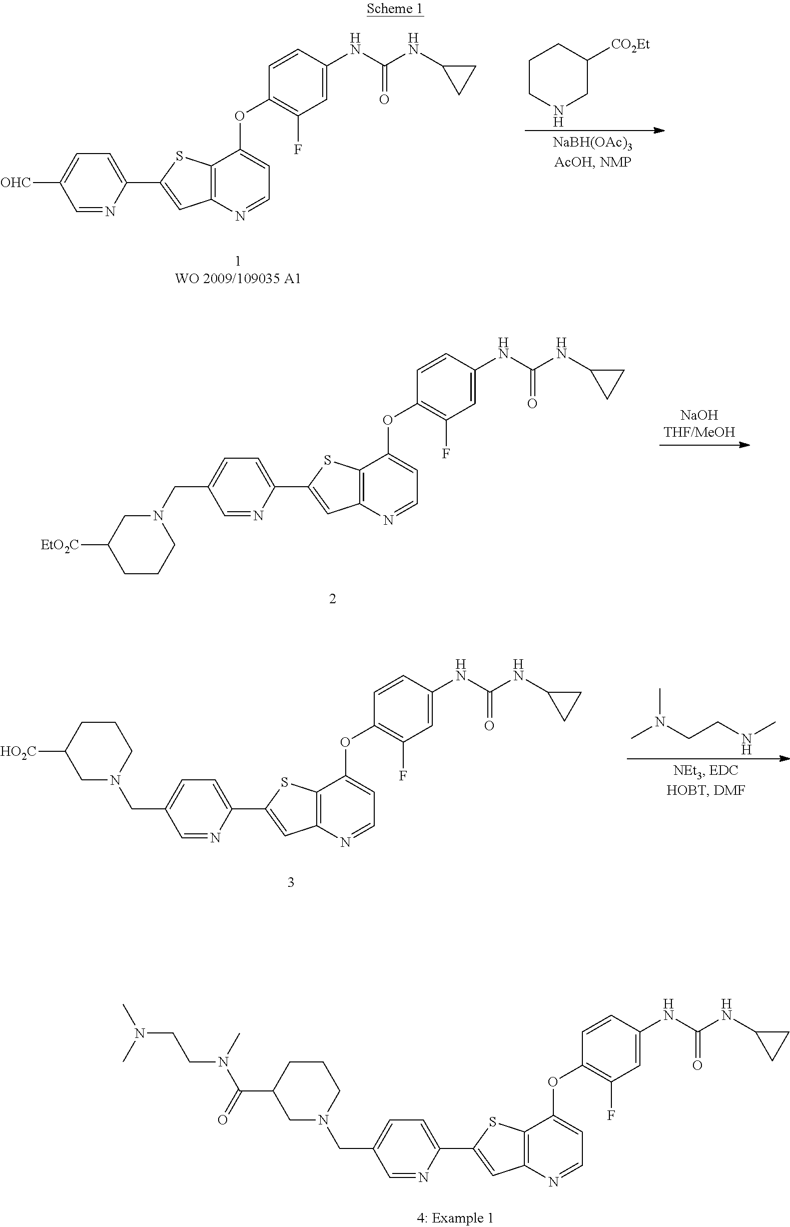

### Example 1

### 1-((6-(7-(4-(3-Cyclopropylureido)-2-fluorophenoxy)thieno [3,2-b]pyridin-2-yl)pyridin-3-yl)methyl)-N-(2-(dimethylamino)ethyl)-N-methylpiperidine-3-carboxamide (4)

Step 1. Ethyl 1-((6-(7-(4-(3-cyclopropylureido)-2-fluorophenoxv)thieno[3,2-b]pyridin-2-yl)pyridin-3-yl)methyl)piperidine-3-carboxylate (2)

To a solution of 1-cyclopropyl-3-(3-fluoro-4-(2-(5-formylpyridin-2-yl)thieno [3,2-b]pyridin-7-yloxy)phenyl)urea (1) (2 g, 4.46 mmol, WO2009/109035 A1) and ethyl nipeconate (1.38 mL, 8.92 mmol) in NMP (40 mL) was added AcOH (0.255 mL, 4.46 mmol). After 30 min, sodium triacetoxyborohydride (2.84 g, 13.38 mmol) was added and the reaction mixture was stirred for 52 h then partitioned between EtOAc and water. The organic layer was collected, washed with water, brine, dried over sodium sulphate, filtered and concentrated. The residue was purified by biotage (SNAP 100g cartridge; MeOH/DCM: 0/100 to 05/95 over 20CV), to afford the title compound 2 that was used in the next step as is. MS (m/z): 590.2 (M+H).

Step 2. 1-((6-(7-(4-(3-Cyclopropylureido)-2-fluorophenoxy)thieno[3,2-b]pyridin-2-yl)pyridin-3-yl)methyl)piperidine-3-carboxylic acid (3)

NaOH 4M (5.58 mL, 22.30 mmol) was added to a solution of crude 2 in a mixture of THF (30 mL) and MeOH (30 mL). The solution was stirred for 1 h then concentrated. Water was added to the residue. After addition of HCl 10% until pH 7, a precipitate was formed that was collected, washed with water and dried under vacuum to afford the title compound 3 (1.93 g, 3.44 mmol, 77% yield over 2 steps) as a red solid. MS (m/z): 562.4 (M+H).

Step 3: 1-((6-(7-(4-(3-Cyclopropylureido)-2-fluorophenoxy)thieno[3,2-b]pyridin-2-yl)pyridin-3-yl)methyl-N-2-dimethylaminoethyl-N-methylpiperidine-3-carboxamide (4)

HOBT (45 mg, 0.294 mmol) was added to a solution of 3 (150 mg, 0.267 mmol), N,N,N′-trimethylethylenediamine (0.069 mL, 0.534 mmol), EDC×HCl (154 mg, 0.801 mmol) and triethylamine (0.149 mL, 1.068 mmol) in DMF (15 mL). The reaction mixture was stirred for 19 h at ambient temperature. The residue was partitioned between EtOAc and water. The organic layer was collected, washed with water, brine, dried over sodium sulphate, filtered and concentrated. The residue was purified by Biotage (SNAP 12g cartridge; MeOH (+2% of NH₄OH)/DCM: 0/100 to 25/75 over 25CV), to afford the title compound 4 (50 mg, 0.077 mmol, 29% yield) as a white solid. ¹H NMR (400 MHz, DMSO-d₆) δ(ppm) 1H: 8.76 (s, 1H), 8.54 (d, J=1.2 Hz, 1H), 8.51 (d, J=5.6 Hz, 1H), 8.32 (s, 1H), 8.23 (d, J=8.4 HZ, 1H), 7.85 (td, J=2.4 and 8.0 Hz, 1H), 7.73 (dd, J=2.4 and 13.6 Hz, 1H), 7.37 (t, J=9.2 Hz, 1H), 7.23-7.18 (m, 1H), 6.64 (d, J=5.6 Hz, 1H), 6.61 (d, J=2.4 Hz, 1H), 3.64-3.24 (m, 3H), 2.97 and 2.75 (s, 3H), 2.87-2.70 (m, 3H), 2.58-2.51 (m, 1H), 2.29-2.08 (m, 2H), 2.10 and 2.08 (s, 6H), 2.06-1.22 (m, 8H), 0.68-0.62 (m, 2H), 0.45-0.40 (m, 2H).MS (m/z): 646.5 (M+H).

Compounds 5-9 (examples 2-6) were prepared similarly to compound 4 (example 1, scheme 1) using the compound 3 as the common intermediate.

TABLE 1
Characterization of compounds 5-9 (examples 2-6)

| Cpd | Ex. | Structure | Characterization |
|---|---|---|---|
| 5 | 2 | 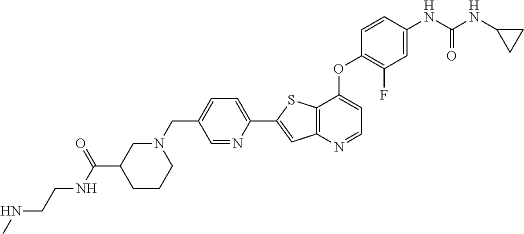 [SMILES: CNCCNC(=O)C1CCCN(Cc2ccc(-c3cc4nccc(Oc5ccc(NC(=O)NC6CC6)cc5F)c4s3)nc2)C1] | ¹H NMR (400 MHz, DMSO-d6) δ (ppm): 8.84 (s, 1H), 8.55 (s, 1H), 8.51 (d, J = 5.6 Hz, 1H), 8.32 (s, 1H), 8.23 (d, J = 8.0 Hz, 1H), 7.85 (dd, J = 2.4 and 8.0 Hz, 1H), 7.79 (t, J = 6.4 Hz, 1H), 7.73 (dd, J = 2.4 and 13.6 Hz, 1H), 7.37 (t, J = 9.2 Hz, 1H), 7.23-7.18 (m, 1H), 6.69 (d, J = 2.4 Hz, 1H), 6.64 (d, J = 5.6 Hz, 1H), 3.56 (d, J = 13.6 Hz, 1H), 3.51 (d, J = 5.6 Hz, 1H), 3.06 (q, J = 6.4 Hz, 2H), 2.75- 2.65 (m, 2H), 2.58-2.51 (m, 1H), 2.47 (t, J = 6.4 Hz, 2H), 2.40-2.30 (m, 1H), 2.23 and 2.05 (s, 3H), 2.12-1.42 (m, 7H), 0.68-0.62 (m, 2H), 0.44-0.40 (m, 2H). MS (m/z): 618.5 (M + 1). |
| | | 1-((6-(7-(4-(3-Cyclopropylureido)-2- | |
| | | fluorophenoxy)thieno[3,2-b]pyridin-2-yl)pyridin- | |
| | | 3-yl)methyl)-N-(2-(methylamino)ethyl)piperidine- | |
| | | 3-carboxamide | |
| 6 | 3 | 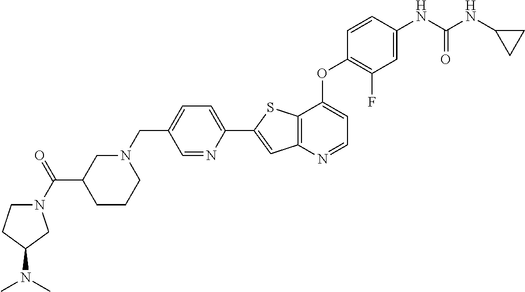 [SMILES: CN(C)[C@H]1CCN(C(=O)C2CCCN(Cc3ccc(-c4cc5nccc(Oc6ccc(NC(=O)NC7CC7)cc6F)c5s4)nc3)C2)C1] | ¹H NMR (400 MHz, DMSO-d₆) δ (ppm): 8.79 (s, 1H), 8.58-8.54 (m, 1H), 8.52 (d, J = 5.6 Hz, 1H), 8.31 (s, 1H), 8.23 (d, J = 8.0 Hz, 1H), 7.89-7.83 (m, 1H), 7.73 (dd, J = 2.4 and 13.6 Hz, 1H), 7.37 (t, J = 9.2 Hz, 1H), 7.24-7.18 (m, 1H), 6.66-6.62 (m, 2H), 3.72-2.51 (m, 12H), 2.14 and 2.11 (s, 6H), 2.09-1.20 (m, 9H), 0.68-0.62 (m, 2H), 0.45- 0.40 (m, 2H). MS (m/z): 658.5 (M + 1). |
| | | 1-Cyclopropyl-3-(4-(2-(5-((3-((S)-3- | |
| | | (dimethylamino)pyrrolidine-1-carbonyl)piperidin- | |
| | | 1-yl)methyl)pyridin-2-yl)thieno[3,2-b]pyridin-7- | |
| | | yloxy)-3-fluorophenyl)urea | |
| 7 | 4 | 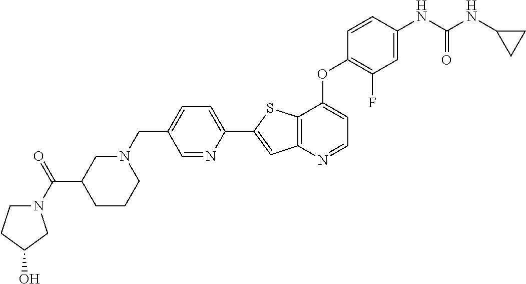 [SMILES: O=C(Nc1ccc(Oc2ccnc3cc(-c4ccc(CN5CCCC(C(=O)N6CC[C@@H](O)C6)C5)cn4)sc23)c(F)c1)NC1CC1] | ¹H NMR (400 MHz, DMSO-d₆) δ (ppm): 8.54 (s, 1H), 8.51 (d, J = 5.6 Hz, 1H), 8.31 (d, J = 1.2 Hz, 1H), 8.23 (d, J = 8.0 Hz, 1H), 7.88-7.83 (m, 1H), 7.76 (dd, J = 2.4 and 13.6 Hz, 1H), 7.37 (t, J = 9.2 Hz, 1H), 6.64 (d, J = 5.6 Hz, 1H), 4.31-4.17 (m, 1H), 3.60-3.18 (m, 6H), 2.85-2.74 (m, 2H), 2.69- 2.51 (m, 2H), 2.10-1.20 (m, 8H), 0.68-0.62 (m, 2H), 0.45-0.40 (m, 2H). MS (m/z): 631.4 (M + 1). |
| | | 1-Cyclopropyl-3-(3-fluoro-4-(2-(5-((3-((R)-3- | |
| | | hydroxypyrrolidine-1-carbonyl)piperidin-1- | |
| | | yl)methyl)pyridin-2-yl)thieno[3,2-b]pyridin-7- | |
| | | yloxy)phenyl)urea | |
| 8 | 5 | 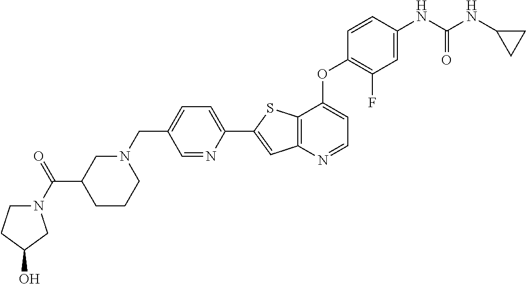 [SMILES: O=C(Nc1ccc(Oc2ccnc3cc(-c4ccc(CN5CCCC(C(=O)N6CC[C@H](O)C6)C5)cn4)sc23)c(F)c1)NC1CC1] | ¹H NMR (400 MHz, DMSO-d₆) δ (ppm): 8.80 (s, 1H), 8.54 (s, 1H), 8.51 (d, J = 5.6 Hz, 1H), 8.32 (s, 1H), 8.23 (d, J = 8.0 Hz, 1H), 7.88-7.83 (m, 1H), 7.73 (dd, J = 2.4 and 13.6 Hz, 1H), 7.38 (t, J = 9.2 Hz, 1H), 7.24-7.18 (m, 1H), 6.65 (s, 1H), 6.64 (d, J = 5.6 Hz, 1H), 5.00-4.86 (m, 1H), 4.30-4.18 (m, 1H), 3.60-3.45 (m, 3H), 3.40-3.15 (m, 2H), 2.85- 2.73 (m, 2H), 2.69-2.51 (m, 2H), 2.11-1.46 (m, 8H), 1.33-1.18 (m, 1H), 0.68-0.62 (m, 2H), 0.45- 0.40 (m, 2H). MS (m/z): 631.4 (M + 1). |
| | | 1-Cyclopropyl-3-(3-fluoro-4-(2-(5-((3-((S)-3- | |
| | | hydroxypyrrolidine-1-carbonyl)piperidin-1- | |
| | | yl)methyl)pyridin-2-yl)thieno[3,2-b]pyridin-7- | |
| | | yloxy)phenyl)urea | |
| 9 | 6 | 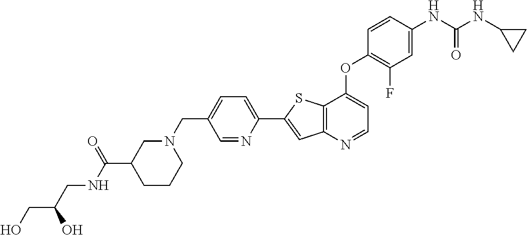 [SMILES: O=C(Nc1ccc(Oc2ccnc3cc(-c4ccc(CN5CCCC(C(=O)NC[C@@H](O)CO)C5)cn4)sc23)c(F)c1)NC1CC1] | ¹H NMR (400 MHz, DMSO-d₆) δ (ppm): 8.80 (s, 1H), 8.55 (s, 1H), 8.51 (d, J = 5.6 Hz, 1H), 8.32 (s, 1H), 8.23 (d, J = 8.0 Hz, 1H), 7.88 (t, J = 5.2 Hz, 1H), 7.86 (dd, J = 2.0 and 8.4 Hz, 1H), 7.73 (dd, J = 2.4 and 13.6 Hz, 1H), 7.38 (t, J = 9.2 Hz, 1H), 7.24-7.18 (m, 1H), 6.65 (s, 1H), 6.64 (d, J = 5.6 Hz, 1H), 4.77-4.72 (m, 1H), 4.56-4.50 (m, 1H), 3.57 (d, J = 13.6 Hz, 1H), 3.51 (d, J = 13.6 Hz, 1H), 3.48-3.40 (m, 1H), 3.30-3.19 (m, 3H), 2.98- 2.92 (m, 1H), 2.77-2.64 (m, 2H), 2.59-2.51 (m, 1H), 2.48-2.38 (m, 1H), 2.18-2.08 (m, 1H), 2.08- 1.97 (m, 1H), 1.72-1.60 (m, 2H), 1.53-1.32 (m, 2H), 0.68-0.62 (m, 2H), 0.45-0.40 (m, 2H). MS (m/z): 635.4 (M + 1). |
| | | 1-((6-(7-(4-(3-Cyclopropylureido)-2-fluoro- | |
| | | phenoxy)thieno[3,2-b]pyridin-2-yl)pyridin-3-yl) | |
| | | methyl)-N-((R)-2,3-dihydroxypropyl)piperidine-3- | |
| | | carboxamide | |

Compounds 10-13 (examples 7-10) were synthesized similarly to compound 4 (example 1, scheme 1) starting from compound 1 and using the corresponding chiral amines instead of racemic ones, in the reductive amination step.

TABLE 2
Characterization of compounds 7-10 (examples 7-10)

| Cpd | Ex. | Structure | Characterization |
|---|---|---|---|
| 10 | 7 | 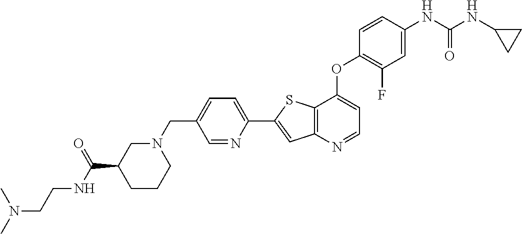 [SMILES: CN(C)CCNC(=O)[C@@H]1CCCN(Cc2ccc(-c3cc4nccc(Oc5ccc(NC(=O)NC6CC6)cc5F)c4s3)nc2)C1] | ¹H NMR (400 MHz, DMSO-d₆) δ (ppm): 8.79 (s, 1H), 8.56 (d, J = 1.2 Hz, 1H), 8.52 (d, J = 5.6 Hz, 1H), 8.33 (s, 1H), 8.24 (d, J = 8.0 Hz, 1H), 7.87 (t, J = 5.6 Hz, 1H), 7.85 (dd, J = 1.6 and 8.0 Hz, 1H), 7.73 (dd, J = 1.6 and 13.2 Hz, 1H), 7.38 (t, J = 8.8 Hz, 1H), 7.24-7.17 (m, 1H), 6.64 (d, J = 5.6 Hz, 1H), 6.61 (d, J = 2.0 Hz, 1H), 3.58 (d, J = 13.6 Hz, 1H), 3.51 (d, J = 13.6 Hz, 1H), 3.18-3.10 (m, 2H), 2.75-2.66 (m, 2H), 2.59-2.52 (m, 1H), 2.45-2.31 (m, 3H), 2.24 (s, 6H), 2.11 (t, J = 10.8 Hz, 1H), 2.02 (t, J = 10.8 Hz, 1H), 1.71-1.60 (m, 2H), 1.55- 1.33 (m, 2H), 0.68-0.62 (m, 2H), 0.45-0.40 (m, 2H). MS (m/z): 632.1 (M + 1). |
| | | (R)-1-((6-(7-(4-(3-Cyclopropylureido)-2- | |
| | | fluorophenoxy)thieno[3,2-b]pyridin-2-yl)pyridin- | |
| | | 3-yl)methyl)-N-(2-(dimethylamino) | |
| | | ethyl)piperidine-3-carboxamide | |
| 11 | 8 | 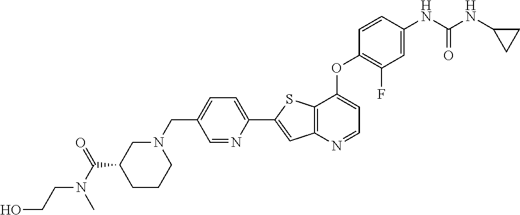 [SMILES: CN(CCO)C(=O)[C@H]1CCCN(Cc2ccc(-c3cc4nccc(Oc5ccc(NC(=O)NC6CC6)cc5F)c4s3)nc2)C1] | ¹H NMR (400 MHz, DMSO-d6) δ (ppm): 8.74 (s, 1H), 8.54 (br s, 1H), 8.52 (d, J = 5.6 Hz, 1H), 8.32 (s, 1H), 8.23 (d, J = 8.0 Hz, 1H), 7.90-7.82 (m, 1H), 7.73 (dd, J = 2.4 and 13.6 Hz, 1H), 7.38 (t, J = 9.2 Hz, 1H), 7.26-7.17 (m, 1H), 6.64 (d, J = 5.6 Hz, 1H), 6.61-6.56 (m, 1H), 4.82 and 4.61 (t, J = 5.6 Hz, 1H), 3.62-3.23 (m, 6H), 3.01 and 2.76 (s, 3H), 2.91-2.72 (m, 3H), 2.58-2.51 (m, 1H), 2.09- 1.88 (m, 2H), 1.80-1.45 (m, 3H), 0.68-0.63 (m, 2H), 0.45-0.40 (m, 2H). MS (m/z): 619.5 (M + 1). |
| | | (S)-1-((6-(7-(4-(3-Ccyclopropylureido)-2- | |
| | | fluorophenoxy)thieno[3,2-b]pyridin-2-yl)pyridin- | |
| | | 3-yl)methyl)-N-(2-hydroxyethyl)-N- | |
| | | methylpiperidine-3-carboxamide | |
| 12 | 9 | 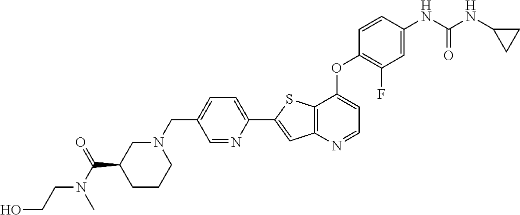 [SMILES: CN(CCO)C(=O)[C@@H]1CCCN(Cc2ccc(-c3cc4nccc(Oc5ccc(NC(=O)NC6CC6)cc5F)c4s3)nc2)C1] | ¹H NMR (400 MHz, DMSO-d₆) δ (ppm): 8.76 (s, 1H), 8.54 (br s, 1H), 8.52 (d, J = 5.6 Hz, 1H), 8.32 (s, 1H), 8.23 (d, J = 8.0 Hz, 1H), 7.90-7.82 (m, 1H), 7.72 (dd, J = 2.4 and 13.6 Hz, 1H), 7.38 (t, J = 9.2 Hz, 1H), 7.24-7.17 (m, 1H), 6.64 (d, J = 5.6 Hz, 1H), 6.60 (d, J = 2.0 Hz, 1H), 4.82 and 4.60 (t, J = 5.2 Hz, 1H), 3.62-3.23 (m, 6H), 3.01 and 2.76 (s, 3H), 2.95-2.70 (m, 3H), 2.58-2.51 (m, 1H), 2.09-1.88 (m, 2H), 1.78-1.45 (m, 3H), 0.68-0.63 (m, 2H), 0.45-0.40 (m, 2H). MS (m/z): 619.5 (M + 1). |
| | | (R)-1-((6-(7-(4-(3-Cyclopropylureido)-2- | |
| | | fluorophenoxy)thieno[3,2-b]pyridin-2-yl)pyridin- | |
| | | 3-yl)methyl)-N-(2-hydroxyethyl)-N- | |
| | | methylpiperidine-3-carboxamide | |
| 13 | 10 | 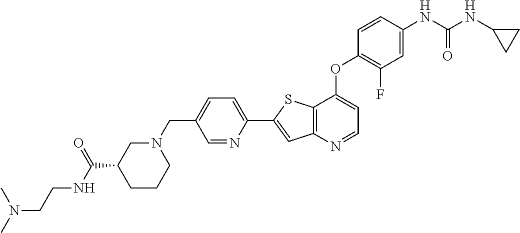 [SMILES: CN(C)CCNC(=O)[C@H]1CCCN(Cc2ccc(-c3cc4nccc(Oc5ccc(NC(=O)NC6CC6)cc5F)c4s3)nc2)C1] | ¹H NMR (400 MHz, DMSO-d₆) δ (ppm): 8.77 (s, 1H), 8.55 (d, J = 1.6 Hz, 1H), 8.52 (d, J = 5.6 Hz, 1H), 8.33 (s, 1H), 8.24 (d, J = 8.4 Hz, 1H), 7.85 (dd, J = 2.4 and 8.0 Hz, 1H), 7.81 (t, J = 5.6 Hz, 1H), 7.73 (dd, J = 2.4 and 13.6 Hz, 1H), 7.38 (t, J = 8.8 Hz, 1H), 7.24-7.18 (m, 1H), 6.66-6.61 (m, 2H), 3.57 (d, J = 13.6 Hz, 1H), 3.49 (d, J = 13.6 Hz, 1H), 3.12-3.06 (m, 2H), 2.73-2.62 (m, 2H), 2.58-2.51 (m, 1H), 2.40-2.31 (m, 1H), 2.20 (t, J = 6.8 Hz, 2H), 2.18-2.00 (m, 2H), 2.10 (s, 6H), 1.70- 1.59 (m, 2H), 1.52-1.33 (m, 2H), 0.68-0.62 (m, 2H), 0.45-0.40 (m, 2H). MS (m/z): 632.1 (M + 1). |
| | | 1 (S)-1-((6-(7-(4-(3-Cyclopropylureido)-2- | |
| | | fluorophenoxy)thieno[3,2-b]pyridin-2-yl)pyridin- | |
| | | 3-yl)methyl)-N-(2-(dimethylamino) | |
| | | ethyl)piperidine-3-carboxamide | |

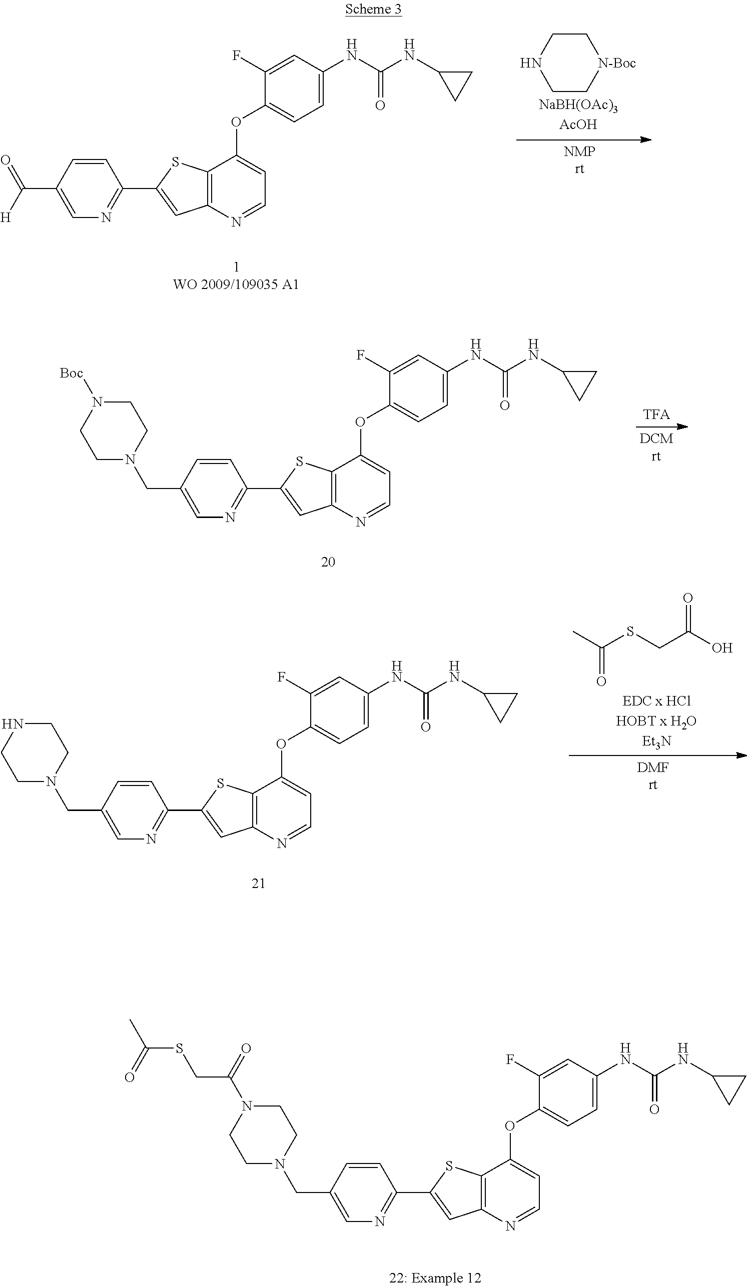

### Example 12

### S-2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl ethanethioate (22)

Step 1. tert-Butyl 4-((6-(7-(4-(3-cyclopropylureido)-2-fluorophenoxy)thieno[3,2-b]-pyridin-2-yl)pyridin-3-yl)methyl)piperazine-1-carboxylate (20)

To a solution of the aldehyde (1) (3.00 g, 6.69 mmol, scheme 1), 1-Boc-piperazine (1.495 g, 8.03 mmol) in NMP (40 ml) at rt under nitrogen were added acetic acid (765 μl, 13.38 mmol) and 15 min later, NaBH(OAc)₃ (4.48 g, 20.07 mmol) portionwise over 2 h. The reaction mixture was stirred at rt overnight, poured into a saturated aqueous sodium bicarbonate solution and stirred for 1 h. The solid was collected by filtration, rinsed with water and dried. The crude product was purified by Biotage (Snap 100 g cartridge; MeOH/DCM: 1/99 to 10/90 over 20 CV), to afford the desired product 20 (3.27 g, 5.29 mmol, 79% yield) as a beige-brown sticky solid (Slightly contaminated by TLC). ¹H NMR (400 MHz, DMSO-d₆) δ (ppm): 8.71 (s, 1H), 8.56 (bd, J=2.0 Hz, 1H), 8.52 (d, J=5.5 Hz, 1H), 8.33 (s, 1H), 8.25 (d, J=8.2 Hz, 1H), 7.87 (dd, J=8.1, 2.1 Hz, 1H), 7.73 (dd, J=13.6, 2.4 Hz, 1H), 7.38 (t, J=9.1 Hz, 1H), 7.20 (bdd, J=8.8, 1.2 Hz, 1H), 6.65 (d, J=5.3 Hz, 1H), 6.57 (bd, J=2.5 Hz, 1H), 3.57 (s, 2H), 4H are hidden by water's peak, 2.59-2.51 (m, 1H), 2.42-2.27 (m, 4H), 1.39 (s, 9H), 0.72-0.58 (m, 2H), 0.50-0.36 (m, 2H). MS (m/z): 619.4 (M+H).

Step 2. 1-Cyclopropyl-3-(3-fluoro-4-(2-(5-(piperazin-1-ylmethyl)pyridin-2-yl)thieno[3,2-b]-pyridin-7-yloxy)phenyl)urea (21)

A solution of compound 20 (3.27 g, 5.29 mmol) and TFA (12.86 ml) in DCM (50 ml) was stirred at rt for 3 h. The reaction mixture was concentrated, diluted with water, stirred for 10 min and poured slowly into a saturated aqueous sodium bicarbonate solution. The pH was adjusted to around 9-10 with 1N NaOH. The resultant suspension was stirred for 1 h, collected by filtration, rinsed with water, and air-dried. The crude material was purified by Biotage (Snap 50 g cartridge; 2% of ammonium hydroxide in MeOH/DCM: 05/95 to 30/70 over 20 CV), to afford the desired product 21 (2.097 g, 3.96 mmol, 75% yield, slightly contaminated with TFA) as a pinky sticky powder which was used in the next step without further purification. ¹H NMR (400 MHz, DMSO-d₆) δ (ppm): 8.76 (bs, 1H), 8.54 (d, J=1.4 Hz, 1H), 8.52 (d, J=5.5 Hz, 1H), 8.32 (s, 1H), 8.24 (d, J=8.2 Hz, 1H), 7.85 (dd, J=8.1, 2.1 Hz, 1H), 7.73 (dd, J=13.5, 2.3 Hz, 1H), 7.38 (t, J=9.1 Hz, 1H), 7.20 (bd, J=10.2 Hz, 1H), 6.64 (d, J=5.5 Hz, 1H), 6.62 (bs, 1H), 3.58-3.48 (m, 2H), 2.73-2.64 (m, 4H), 2.59-2.52 (m, 1H), 2.38-2.25 (m, 4H), 0.69-0.62 (m, 2H), 0.46-0.40 (m, 2H), one NH is missing. MS (m/z): 519.6 (M+H).

Step 3. S-2-(4-((6-(7-(4-(3-Cyclopropylureido)-2-fluorophenoxy)thieno[3,2-b]pyridin-2-yl)pyridin-3-yl)methyl)piperazin-1-D-2-oxoethyl ethanethioate (22)

A stirred solution of compound 21 (150 mg, 0.28 mmol), 2-(acetylthio)acetic acid (113 mg, 0.84 mmol) and triethylamine (156 μl, 1.12 mmol) in DMF (10 ml) under nitrogen was sonicated for 2 hrs. HOBT-Monohydrate (52 mg, 0.34 mmol) and EDC-hydrochloride (161 mg, 0.84 mmol) were added and the reaction mixture, was stirred at RT overnight. The reaction mixture was diluted with AcOEt and successively washed with a saturated aqueous solution of sodium bicarbonate, water and brine, dried over anhydrous magnesium sulfate, filtered and concentrated. The residue was purified twice by Biotage (Snap 25 g cartridge; MeOH/DCM: 1/99 to 10/90 over 30 CV), to afford the desired product 22 (63 mg, 0.1 mmol, 35% yield) as an off-white sticky solid. ¹H NMR (400 MHz, DMSO-d₆) δ (ppm): 8.71 (s, 1H), 8.58 (bd, J=1.6 Hz, 1H), 8.52 (d, J=5.3 Hz, 1H), 8.34 (s, 1H), 8.26 (d, J=8.0 Hz, 1H), 7.88 (dd, J=8.1, 2.1 Hz, 1H), 7.73 (dd, J=13.6, 2.4 Hz, 1H), 7.38 (t, J=9.1 Hz, 1H), 7.20 (dd, J=8.8, 1.4 Hz, 1H), 6.65 (dd, J=5.5, 0.6 Hz, 1H), 6.57 (bd, J=2.5 Hz, 1H), 3.88 (s, 2H), 3.60 (s, 2H), 3.55-3.41 (m, 4H), 2.59-2.51 (m, 1H), 2.47-2.42 (m, 2H), 2.39-2.33 (m, 5H), 0.72-0.58 (m, 2H), 0.50-0.36 (m, 2H). MS (m/z): 635.5 (M+H).

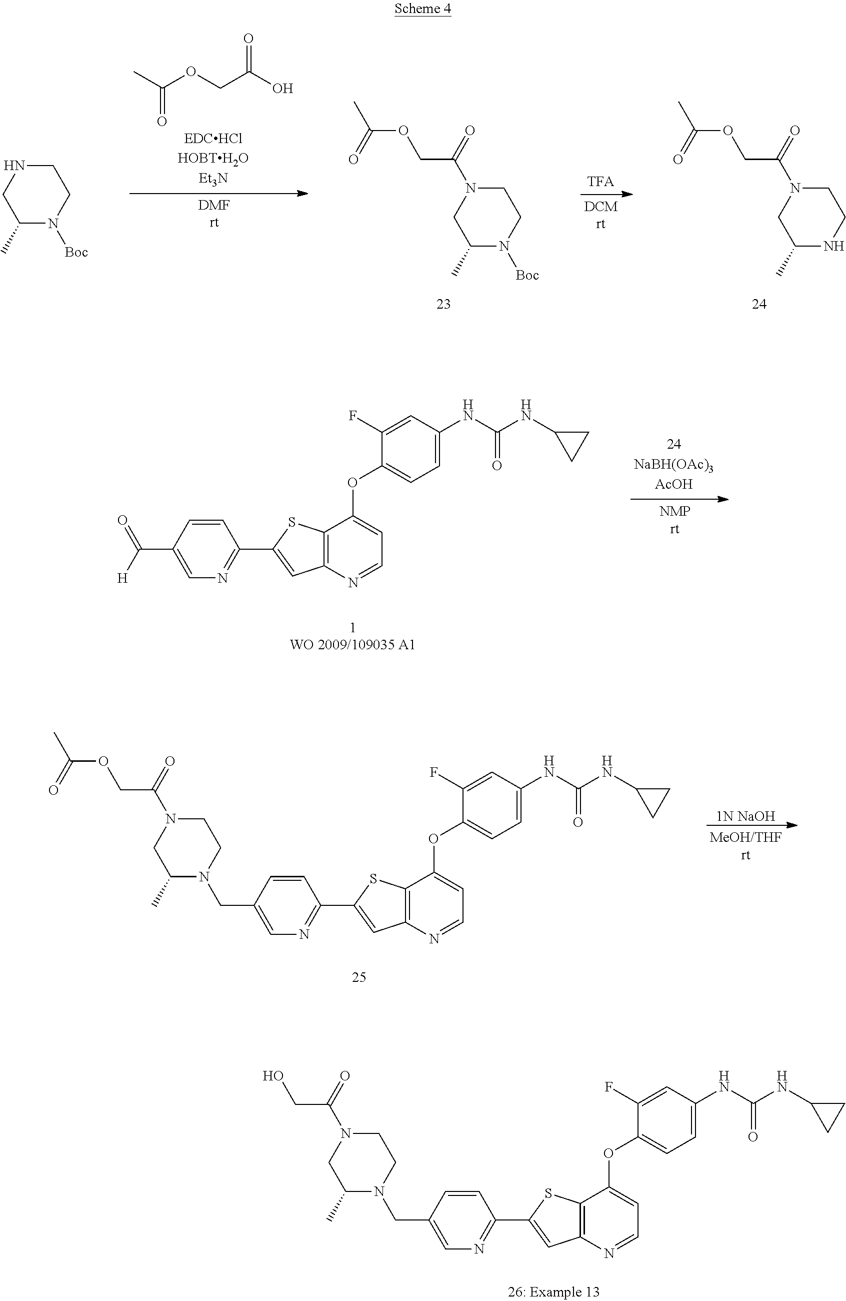

### Example 13

### (R)-1-Cyclopropyl-3-(3-fluoro-4-(2(5-((44-(2-hydroxyacetyl)-2-methylpiperazin-1-yl)methyl)pyridin-2-yl)thieno[3,2-b]pyridin-7-yloxy)phenyl)urea (26)

Step 1: (R)-tert-Butyl 4-(2-acetoxyacetyl)-2-methylpiperazine-1-carboxylate (23)

To stirred solution of (R)-N-Boc-2-methylpiperazine (500 mg, 2.5 mmol), acetoxyacetic acid (537 mg, 4.54 mmol) and triethylamine (1.26 ml, 9.11 mmol) in DMF (10 ml) under nitrogen were added HOBT-monohydrate (382 mg, 2.5 mmol) and EDC-hydrochloride (1.316 g, 6.87 mmol), and the reaction mixture was stirred at rt overnight. The reaction mixture was diluted with AcOEt and successively washed with a saturated aqueous sodium bicarbonate solution, a saturated aqueous ammonium chloride solution, water and brine, dried over anhydrous magnesium sulfate, filtered, and concentrated to afford the desired product 23 (786 mg, quantitative yield) as a pale-yellow sticky oil. The crude product was used in the next step without any further purification. MS (m/z): 323.3 (M+Na).

Step 2: (R)-2-(3-Methylpiperazin-1-yl)-2-oxoethyl acetate (24)

A solution of compound 23 (crude, 2.497 mmol) and TFA (10 ml) in DCM (25 ml) was stirred at rt for 4 h. The reaction mixture was concentrated, diluted in water, poured slowly into a saturated aqueous sodium bicarbonate solution (200 ml) and extracted with DCM (with traces of methanol). The combined extract was dried over anhydrous magnesium sulfate, filtered, and concentrated. The residue was purified by Biotage (Snap 10 g cartridge; 2% of ammonium hydroxide in MeOH/DCM: 01/99 to 15/85 over 20 CV), to afford the desired product 24 (226 mg, 1.13 mmol, 45% yield) as a colorless sticky oil. MS (m/z): 200.95 (M+H).

Step 3: (R)-2-(4-((6-(7-(4-(3-Cyclopropylureido)-2-fluorophenoxy)thieno[3,2-b]pyridin-2-yl)pyridin-3-yl)methyl)-3-methylpiperazin-1-yl)-2-oxoethyl acetate (25)

To a solution of the aldehyde 1 (400 mg, 0.89 mmol, scheme 1), compound 24 (223 mg, 1.11 mmol) and acetic acid (102 μl, 1.78 mmol) in NMP (20 ml) at rt under nitrogen was added NaBH(OAc)₃ (597 mg, 2.68 mmol) portionwise over 1 h. The reaction mixture was stirred at rt overnight, diluted with water, poured into a saturated aqueous sodium bicarbonate solution and stirred for 1 h. A precipitate was formed which was collected by filtration, rinsed with water and air-dried. The crude product was adsorbed on silica gel and purified twice by Biotage (Snap 25 g cartridge; MeOH/DCM: 1/99 to 10/90 over 30 CV; Snap 100 g cartridge: MeOH/DCM: 1/99 to 10/90 over 30 CV), to afford the desired product 25 (154 mg, 0.24 mmol, 27% yield) as a colorless-pale orange sticky film. MS (m/z): 633.4 (M+H).

Step 4: (R)-1-Cyclopropyl-3-(3-fluoro-4-(2-(5-((4-(2-hydroxyacetyl)-2-methyl piperazin-1-yl)methyl)pyridin-2-yl)thieno[3,2-b]pyridin-7-yloxy)phenyl)urea (26)

To a solution of compound 25 (154 mg, 0.24 mmol) in a mixture of MeOH/THF (5/5 ml) was added 1N NaOH (2.43 ml). The reaction mixture was stirred at rt for 1 h, concentrated, diluted with MeOH, sonicated for 15 min, diluted with water and sonicated for an additional 30 min. A precipitate was formed which was collected by filtration, rinsed with water and dried. The crude product was purified by Biotage (Snap 25 g cartridge; 2% of ammonium hydroxide in MeOH/DCM: 1/99 to 15/85 over 30 CV), to afford the desired product 26 (92 mg, 0.156 mmol, 64% yield) as an off-white fluffy solid. ¹H NMR (400 MHz, DMSO-d₆) δ (ppm): 8.72 (s, 1H), 8.58 (bd, J=1.8 Hz, 1H), 8.52 (d, J=5.5 Hz, 1H), 8.33 (s, 1H), 8.25 (d, J=8.0 Hz, 1H), 7.88 (dd, J=8.1, 2.1 Hz, 1H), 7.73 (dd, J=13.6, 2.4 Hz, 1H), 7.38 (t, J=9.1 Hz, 1H), 7.20 (dd, J=8.9, 1.3 Hz, 1H), 6.65 (dd, J=5.4, 0.7 Hz, 1H), 6.58 (bd, J=2.5 Hz, 1H), 4.56 (q, J=5.3 Hz, 1H), 4.16-3.99 (m, 2H), 3.96 (d, J=14.1 Hz, 1H), 3.88-3.74 (m, 1H), 3.56-3.34 (m, 2H), 3.22-3.02 (m, 1H), 3.00-2.86 (m, 1H), 2.69-2.60 (m, 1H), 2.59-2.51 (m, 1H), 2.50-2.40 (m, 1H), 2.22-2.04 (m, 1H), 1.13-1.06 (m, 3H), 0.72-0.58 (m, 2H), 0.49-0.36 (m, 2H). MS (m/z): 591.4 (M+H).

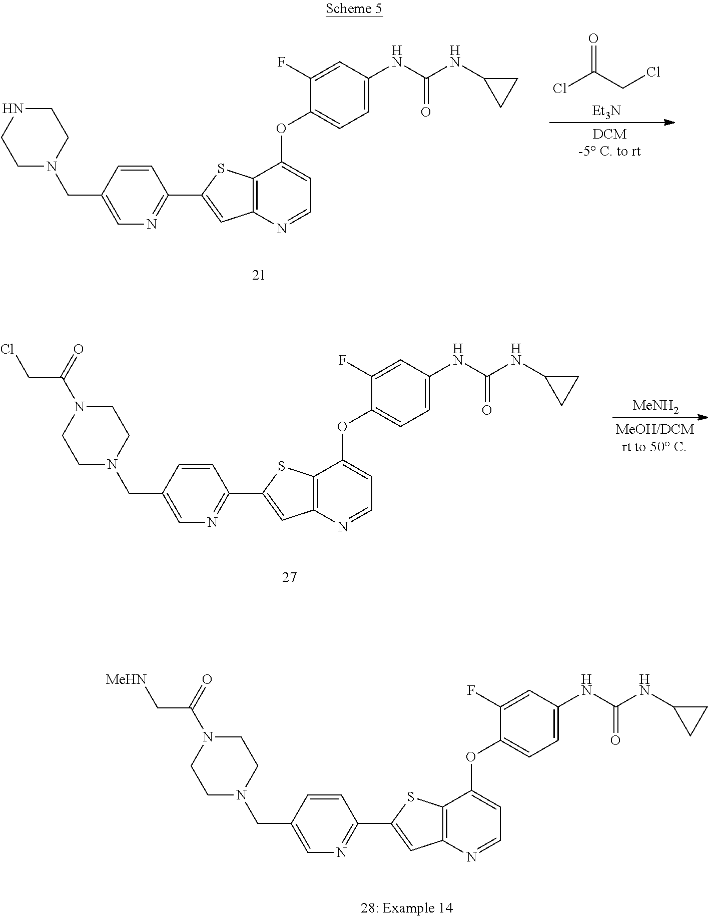

### Example 14

1-Cyclopropyl-3-(3-fluoro-4-(2-(5-((4-(2-(methylamino)acetyl)piperazin-1-yl)methyl)pyridin-2-yl)thieno[3,2-b]pyridin-7-yloxy)phenyl)urea (28)

Step 1: 1-(4-(2-(5-((4-(2-Chloroacetyl)piperazin-1-yl)methyl)pyridin-2-yl)thieno [3,2-b]pyridin-7-yloxy)-3-fluorophenyl)-3-cyclopropylurea (27)

To a stirred suspension at −5° C. of compound 21 (300 mg, 0.58 mmol, scheme 3) and triethylamine (241 μl, 1.73 mmol) in DCM (30 ml) under nitrogen was slowly added chloroacetyl chloride (61 μl, 0.75 mmol). The reaction mixture was allowed to warm-up to rt over 1 h, and was stirred at rt for 15 min. The reaction was quenched by addition of methanol; the mixture was concentrated and diluted with AcOEt. The resultant solution was successively washed with a saturated aqueous sodium bicarbonate solution, a saturated aqueous ammonium chloride solution, water and brine, dried over anhydrous magnesium sulfate, filtered and evaporated, to afford the desired product 27 (370 mg, quantitative yield) as an yellow sticky foam. The material was used in the next step without any further purification. MS (m/z): 595.5-597.5 (M+H).

Step 2: 1-Cyclopropyl-3-(3-fluoro-4-(2-(5-((4-(2-(methylamino)acetyl)piperazin-1-yl)methyl)pyridin-2-yl)thieno[3,2-b]pyridin-7-yloxy)phenyl)urea (28)

To a stirred solution of compound 27 (250 mg, 0.42 mmol) in a mixture of MeOH/DCM (10 ml/10 ml) under nitrogen was added a solution of methylamine in methanol (2.1 ml), and the reaction mixture was stirred at rt overnight (almost no reaction by MS). More methylamine was added (4.2 ml) and the reaction mixture was heated at 50° C. for 4 h, then rt and concentrated. The crude residue was purified by Biotage (Snap 25 g cartridge; 2% of ammonium hydroxide in MeOH/DCM: 1/99 to 20/80 over 30 CV). The desired fractions were combined, concentrated, and dried under high vacuum to afford the desired product 28 (162 mg, 0.26 mmol, 63% yield) as a beige sticky solid (ammonium salt). ¹H NMR (400 MHz, DMSO-d₆) δ (ppm): 9.15 (bs, 1H), 9.00-8.66 (m, 1.6H), 8.59 (bs, 1H), 8.52 (d, J=5.5 Hz, 1H), 8.35 (s, 1H), 8.27 (bd, J=8.2 Hz, 1H), 7.88 (bd, J=7.8 Hz, 1H), 7.73 (dd, J=13.6, 2.4 Hz, 1H), 7.50-7.00 (m, 5H), 6.79 (d, J=2.7 Hz, 1H), 6.65 (d, J=4.9 Hz, 1H), 4.03 (s, 2H), 3.63 (bs, 2H), 3.52 (bs, 2H), 3.37 (bs, 2H), 2.59-2.51 [m, 1H, overlapped with a singlet at 2.54 (s, 3H)], 2.50-2.30 (m, 4H), 0.71-0.58 (m, 2H), 0.48-0.35 (m, 2H). MS (m/z): 590.5 (M+H).

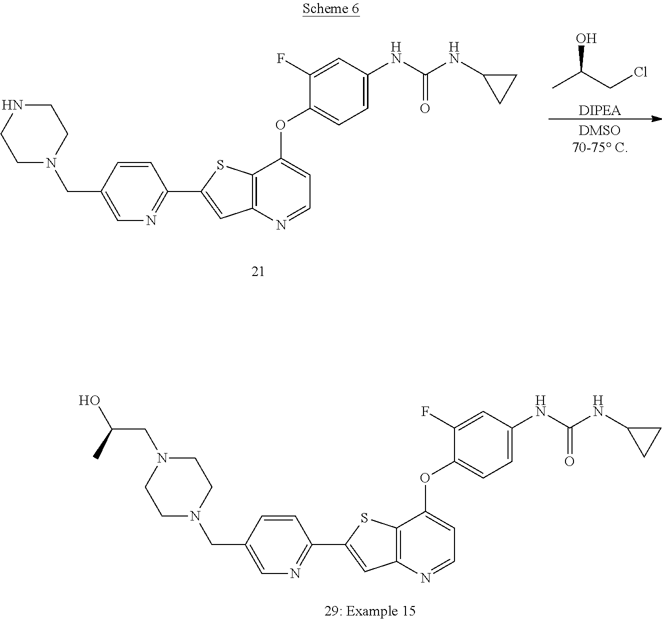

### Example 15

(R)-1-Cyclopropyl-3-(3-fluoro-4-(2-(5-((4-(2-hydroxypropyl)piperazin-1-yl)methyl)pyridin-2-yl)thieno[3,2-b]pyridin-7-yloxy)phenyl)urea (29)

To a stirred solution of compound 21 (200 mg, 0.39 mmol, scheme 3) and DIPEA (269 μl, 1.54 mmol) in DMSO (10 ml) under nitrogen at rt was added (R)-(−)-1-chloro-2-propanol (657 μl, 7.71 mmol, ee=99.2%), and the reaction mixture was heated at 70° C. overnight. More (R)-(−)-1-chloro-2-propanol (657 μl, 7.71 mmol) was added and the reaction mixture was heated at 75° C. overnight. The reaction mixture was then cooled to rt, diluted with AcOEt, and successively washed with a saturated aqueous sodium bicarbonate solution, water and brine, dried over anhydrous magnesium sulfate, filtered and concentrated. The residue was purified by Biotage (Snap 25 g cartridge; 2% of ammonium hydroxide in MeOH/DCM: 1/99 to 15/85 over 30 CV), to afford the desired product 29 (59 mg, 0.10 mmol, 26% yield) as an ivory-colored sticky solid. ¹H NMR (400 MHz, DMSO-d₆) δ (ppm): 8.71 (s, 1H), 8.54 (bd, J=1.2 Hz, 1H), 8.52 (d, J=5.5 Hz, 1H), 8.32 (s, 1H), 8.24 (d, J=8.0 Hz, 1H), 7.85 (dd, J=8.1, 2.1 Hz, 1H), 7.73 (dd, J=13.6, 2.4 Hz, 1H), 7.38 (t, J=9.1 Hz, 1H), 7.20 (dd, J=9.0, 1.2 Hz, 1H), 6.64 (d, J=5.5 Hz, 1H), 6.57 (bd, J=1.8 Hz, 1H), 4.38-4.12 (m, 1H), 3.81-3.66 (m, 1H), 3.55 (s, 2H), 2.60-2.52 (m, 1H), 2.50-2.32 (m, 8H), 2.30-2.08 (m, 2H), 1.02 (d, J=6.1 Hz, 3H), 0.72-0.58 (m, 2H), 0.50-0.36 (m, 2H). MS (m/z): 577.5 (M+H).

Compound 30 (example 16) was prepared in one step by reacting compound 21 with (S)-1-chloro-2-propanol similarly to compound 29 (scheme 6). Compound 31 (example 17) was prepared in one step by reacting compound 122 (scheme 28) with 2-(2-(2-methoxyethoxy)ethoxy)acetic acid using the procedure similar to the one described above for the synthesis of compound 22 (scheme 3).

TABLE 3
Characterization of compounds 30-31 (example 16-17)

| Cpd | Ex. | Structure | Characterization |
|---|---|---|---|
| 30 | 16 | 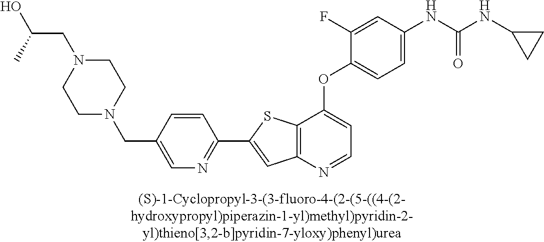 [SMILES: C[C@H](O)CN1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.71 (s, 1H), 8.54 (bd, J = 1.4 Hz, 1H), 8.52 (d, J = 5.3 Hz, 1H), 8.32 (s, 1H), 8.24 (dd, J = 8.2, 0.6 Hz, 1H), 7.85 (dd, J = 8.1, 2.1 Hz, 1H), 7.73 (dd, J = 13.6, 2.4 Hz, 1H), 7.38 (t, J = 9.1 Hz, 1H), 7.20 (dd, J = 9.0, 1.4 Hz, 1H), 6.64 (dd, J = 5.3, 0.8 Hz, 1H), 6.57 (bd, J = 2.5 Hz, 1H), 4.34-4.16 (m, 1H), 3.79-3.67 (m, 1H), 3.54 (s, 2H), 2.59-2.51 (m, 1H), 2.49- 2.30 (m, 8H), 2.28-2.10 (m, 2H), 1.02 (d, J = 6.3 Hz, 3H), 0.72-0.58 (m, 2H), 0.50-0.36 (m, 2H). MS (m/z): 577.5 (M + H). |
| 31 | 17 | 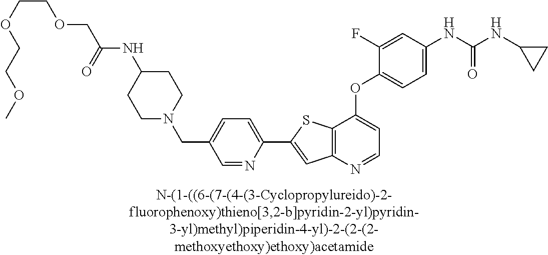 [SMILES: COCCOCCOCC(=O)NC1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.88 (s, 1H), 8.54 (brd, J = 1.2 Hz, 1H), 8.51 (d, J = 5.2 Hz, 1H), 8.33 (s, 1H), 8.24 (d, J = 8.0 Hz, 1H), 7.84 (dd, J = 8.0, 2.0 Hz, 1H), 7.73 (dd, J = 13.6, 2.4 Hz, 1H), 7.47 (d, J = 8.4 Hz, 1H), 7.38 (t, J = 9.0 Hz, 1H), 7.23-7.18 (m, 1H), 6.73 (brd, J = 2.4 Hz, 1H), 6.64 (dd, J = 5.2, 0.8 Hz, 1H), 3.85 (s, 2H), 3.66-3.56 (m, 1H), 3.56-3.52 (m, 8H), 3.47-3.44 (m, 2H), 3.25 (s, 3H), 2.83-2.76 (m, 2H), 2.58-2.51 (m, 1H), 2.11-2.03 (m, 2H), 1.73-1.67 (m, 2H), 1.57-1.44 (m, 2H), 0.68-0.62 (m, 2H), 0.45- 0.40 (m, 2H). MS (m/z): 693.69 (M + H). |

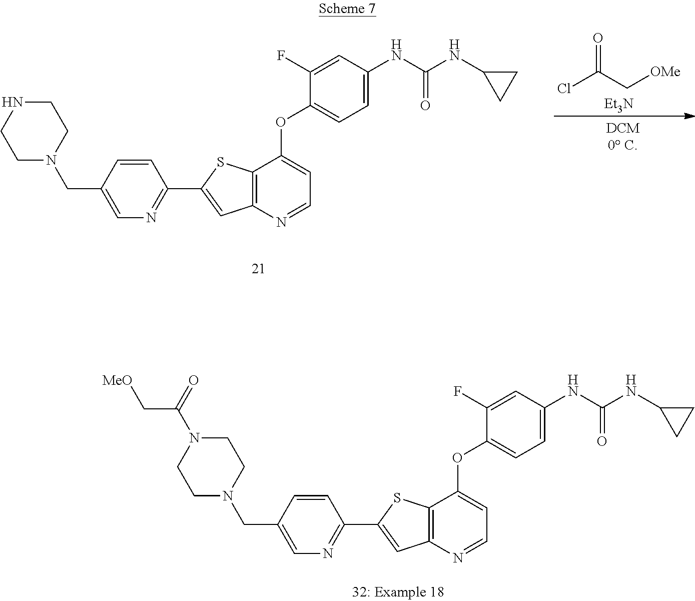 [SMILES: "Et3N" ; O=C(Nc1ccc(Oc2ccnc3cc(-c4ccc(CN5CCNCC5)cn4)sc23)c(F)c1)NC1CC1 ; COCC(=O)Cl ; COCC(=O)N1CCN(Cc2ccc(-c3cc4nccc(Oc5ccc(NC(=O)NC6CC6)cc5F)c4s3)nc2)CC1]

### Example 18

### 1-Cyclopropvl-3-(3-fluoro-4-(2-(5-((4-(2-methoxyacetyl)piperazin-1-yl)methyl)pyridin-2-yl)thieno[3,2-b]pyridin-7-yloxy)phenyl)urea (32)

To a stirred suspension of compound 21 (150 mg, 0.29 mmol) and triethylamine (160 μl, 1.16 mmol) in DCM (15 ml) at 0° C. under nitrogen was slowly added methoxyacetyl chloride (53 μl, 0.58 mmol). The reaction mixture was stirred at 0° C. for 1 h, quenched by addition of methanol, concentrated, diluted with AcOEt and successively washed with a saturated aqueous solution of sodium bicarbonate, a saturated aqueous solution of ammonium chloride, water and brine, dried over anhydrous magnesium sulfate, filtered, and concentrated. The residue was purified by Biotage (Snap 25 g cartridge; MeOH/DCM: 1/99 to 10/90 over 30 CV), to afford the desired product 32 (86 mg, 0.146 mmol, 50% yield) as an off-white sticky solid. ¹H NMR (400 MHz, DMSO-d₆) δ (ppm): 8.73 (s, 1H), 8.57 (bd, J=1.8 Hz, 1H), 8.52 (d, J=5.5 Hz, 1H), 8.34 (s, 1H), 8.25 (d, J=8.0 Hz, 1H), 7.88 (dd, J=8.2, 2.0 Hz, 1H), 7.73 (dd, J =13.5, 2.5 Hz, 1H), 7.38 (t, J=9.0 Hz, 1H), 7.20 (dd, J=8.8, 1.4 Hz, 1H), 6.65 (d, J=4.9 Hz, 1H), 6.59 (bd, J=2.3 Hz, 1H), 4.06 (s, 2H), 3.59 (s, 2H), 3.50-3.35 (m, 4H), 3.27 (s, 3H), 2.59-2.51 (m, 1H), 2.48-2.36 (m, 4H), 0.72-0.58 (m, 2H), 0.50-0.36 (m, 2H). MS (m/z): 591.4 (M+H).

Compound 33 (example 19) was prepared in four steps starting from aldehyde 1 and (R)-1-N-Boc-2-methyl piperazine, and using procedures similar to the ones described in the scheme 4 for the synthesis of compound 26 (example 13). Compounds 34-36 (examples 20-22) were obtained starting from compound 21 and using the procedures similar to the one described above for the synthesis of compound 22 (example 12, scheme 3).

TABLE 4
Characterization of compounds 33-36 (example 19-22).

| Cpd | Ex. | Structure | Characterization |
|---|---|---|---|
| 33 | 19 | 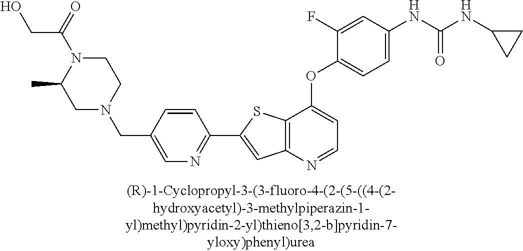 [SMILES: C[C@@H]1CN(Cc2ccc(-c3cc4nccc(Oc5ccc(NC(=O)NC6CC6)cc5F)c4s3)nc2)CCN1C(=O)CO] | ¹H NMR (400 MHz, DMSO-d₆) δ (ppm): mixture of rotamers, 8.77 (s, 1H), 8.58 (bd, J = 1.6 Hz, 1H), 8.52 (d, J = 5.5 Hz, 1H), 8.35 (s, 1H), 8.27 (d, J = 8.2 Hz, 1H), 7.89 (dd, J = 8.1, 2.1 Hz, 1H), 7.73 (dd, J = 13.6, 2.4 Hz, 1H), 7.38 (t, J = 9.1 Hz, 1H), 7.20 (bd, J = 8.8 Hz, 1H), 6.70-6.58 (m, 2H), 4.58-3.40 (m, 7H), 3.30-2.74 (m, 2H), 2.65 (bd, J = 11.9 Hz, 1H), 2.59-2.51 (m, 1H), 2.22-1.84 (m, 2H), 1.38-1.10 (m, 3H), 0.72-0.58 (m, 2H), 0.50-0.36 (m, 2H).. MS (m/z): 591.4 (M + H). |
| 34 | 20 | 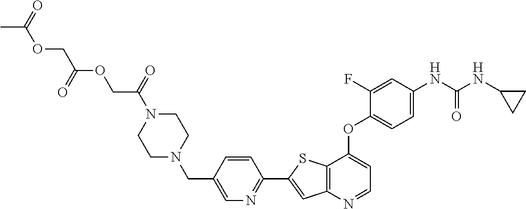 [SMILES: CC(=O)OCC(=O)OCC(=O)N1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.74 (s, 1H), 8.57 (bd, J = 1.6 Hz, 1H), 8.51 (d, J = 5.5 Hz, 1H), 8.35 (s, 1H), 8.27 (d, J = 8.2 Hz, 1H), 7.88 (dd, J = 8.2, 2.0 Hz, 1H), 7.73 (dd, J = 13.6, 2.4 Hz, 1H), 7.39 (t, J = 9.1 Hz, 1H), 7.20 (bd, J = 8.8 Hz, 1H), 6.65 (dd, J = 5.5, 0.8 Hz, 1H), 6.61 (bs, 1H), 4.89 (s, 2H), 4.73 (s, 2H), 3.60 (s, 2H), 3.48-3.35 (m, 4H), 2.59-2.51 (m, 1H), 2.47-2.33 (m, 4H), 2.10 (s, 3H), 0.72-0.58 (m, 2H), 0.49-0.36 (m, 2H). MS (m/z): 677.5 (M + H). |
| | | 2-(4-((6-(7-(4-(3-Cyclopropylureido)-2- | |
| | | fluorophenoxy)thieno[3,2-b]pyridin-2-yl)pyridin-3- | |
| | | yl)methyl)piperazin-1-yl)-2-oxoethyl 2- | |
| | | acetoxyacetate | |
| 35 | 21 | 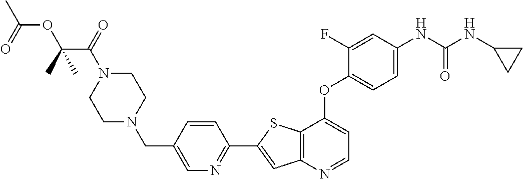 | ¹H NMR (400 MHz, DMSO-d₆) δ (ppm): 8.74 (s, 1H), 8.57 (bd, J = 1.6 Hz, 1H), 8.52 (d, J = 5.5 Hz, 1H), 8.34 (s, 1H), 8.25 (d, J = 8.0 Hz, 1H), 7.88 (dd, J = 8.2, 2.2 Hz, 1H), 7.73 (dd, J = 13.5, 2.5 Hz, 1H), 7.38 (t, J = 9.0 Hz, 1H), 7.20 (dd, J = 8.9, 1.3 Hz, 1H), 6.65 (dd, J = 5.4, 0.7 Hz, 1H), 6.60 (bd, J = 2.2 Hz, 1H), 3.66-3.40 (m, 6H), 2.59-2.51 (m, 1H), 2.42-2.28 (m, 4H), 2.04 (s, 3H), 1.46 (s, 6H), 0.72-0.58 (m, 2H), 0.49-0.36 (m, 2H). MS (m/z): 647.55 (M + H). |
| | | 1-(4-((6-(7-(4-(3-Cyclopropylureido)-2- | |
| | | fluorophenoxy)thieno[3,2-b]pyridin-2-yl)pyridin-3- | |
| | | yl)methyl)piperazin-1-yl)-2-methyl-1-oxopropan-2- | |
| | | yl acetate | |
| 35-A | 21-A | 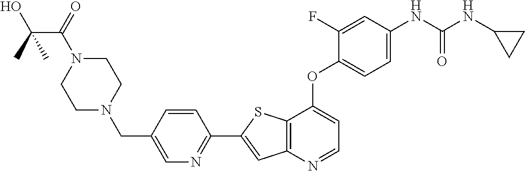 | ¹H NMR (400 MHz, DMSO-d₆) δ (ppm): 8.74 (s, 1H), 8.57 (bd, J = 1.6 Hz, 1H), 8.52 (d, J = 5.5 Hz, 1H), 8.34 (s, 1H), 8.25 (d, J = 8.0 Hz, 1H), 7.88 (dd, J = 8.2, 2.2 Hz, 1H), 7.73 (dd, J = 13.5, 2.5 Hz, 1H), 7.38 (t, J = 9.0 Hz, 1H), 7.20 (dd, J = 8.9, 1.3 Hz, 1H), 6.65 (dd, J = 5.4, 0.7 Hz, 1H), 6.60 (bd, J = 2.2 Hz, 1H), 3.66-3.40 (m, 6H), 2.59-2.51 (m, 1H), 2.42-2.28 (m, 4H), 2.04 (s, 3H), 1.46 (s, 6H), 0.72-0.58 (m, 2H), 0.49-0.36 (m, 2H). MS (m/z): 647.55 (M + H). |
| | | 1-Cyclopropyl-3-(3-fluoro-4-(2-(5-((4-(2-hydroxy- | |
| | | 2-methylpropanoyl)piperazin-1-yl)methyl)pyridin- | |
| | | 2-yl)thieno[3,2-b]pyridin-7-yloxy)phenyl)urea | |
| 36 | 22 | 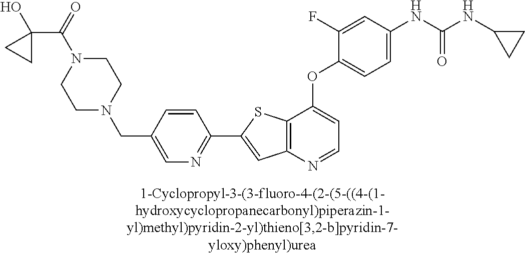 [SMILES: O=C(Nc1ccc(Oc2ccnc3cc(-c4ccc(CN5CCN(C(=O)C6(O)CC6)CC5)cn4)sc23)c(F)c1)NC1CC1] | ¹H NMR (400 MHz, DMSO-d₆) δ (ppm): 8.72 (s, 1H), 8.57 (bd, J = 1.4 Hz, 1H), 8.52 (d, J = 5.5 Hz, 1H), 8.34 (s, 1H), 8.26 (d, J = 8.0 Hz, 1H), 7.88 (dd, J = 8.1, 2.0 Hz, 1H), 7.73 (dd, J = 13.6, 2.4 Hz, 1H), 7.38 (t, J = 9.1 Hz, 1H), 7.20 (bd, J = 8.8 Hz, 1H), 6.65 (dd, J = 5.4, 0.7 Hz, 1H), 6.58 (bd, J = 2.5 Hz, 1H), 6.44 (s, 1H), 6.36 (s, 1H), 4.33-4.27 (m, 1H), 4.16-4.10 (m, 1H), 3.59 (s, 2H), 3.52-3.40 (m, 4H), 3.13-3.06 (m, 1H), 2.82 (dd, J = 12.4, 5.0 Hz, 1H), 2.61- 2.51 (m, 2H), 2.44-2.25 (m, 6H), 1.67-1.25 (m, 6H), 0.72-0.58 (m, 2H), 0.50-0.37 (m, 2H). MS (m/z): 745.7 (M + H). |

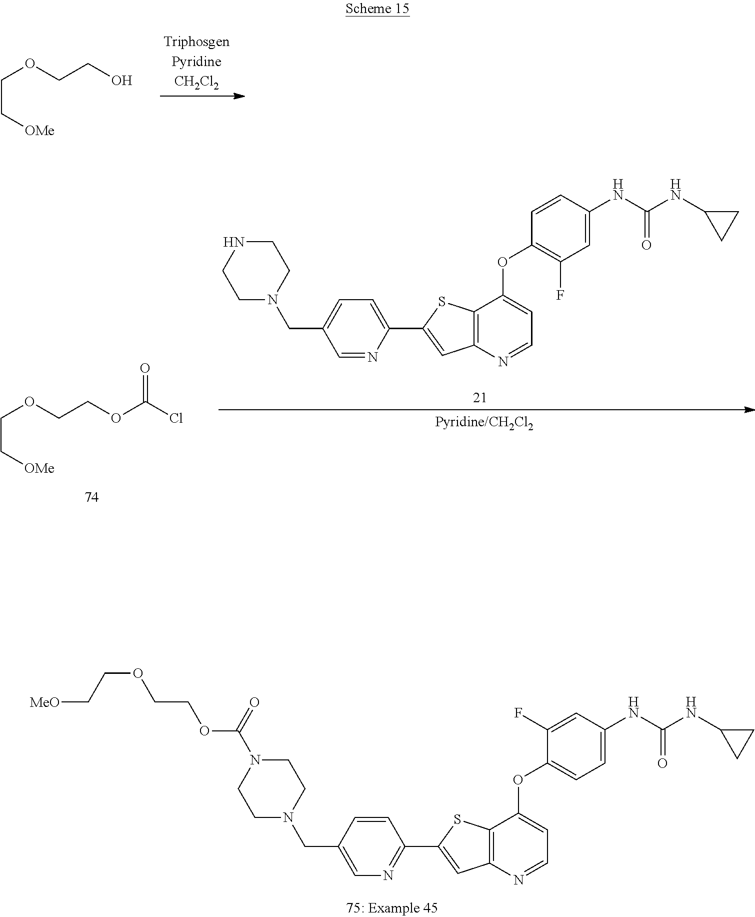 [SMILES: COCCOCCO ; O=C(Nc1ccc(Oc2ccnc3cc(-c4ccc(CN5CCNCC5)cn4)sc23)c(F)c1)NC1CC1 ; COCCOCCOC(=O)Cl ; COCCOCCOC(=O)N1CCN(Cc2ccc(-c3cc4nccc(Oc5ccc(NC(=O)NC6CC6)cc5F)c4s3)nc2)CC1]

### Example 45

### 2-(2-Methoxyethoxy)ethyl 4-((6-(7-(4-(3-cyclopropylureido)-2-fluorophenoxy)thieno[3,2-b]pyridin-2-yl)pyridin-3-yl)methyl)piperazine-1-carboxylate (75)

To a solution of 2-(2-methoxyethoxy)ethanol (0.24 g, 2.0 mmol) and triphosgen (0.21 g, 0.71 mmol) in DCM (10 mL) was added dropwise pyridine (0.19 g, 2.4 mmol), and the resultant mixture was stirred at room temperature for 2 hours to afford the 2-(2-methoxyethoxy)ethyl carbonochloridate (74) in a DCM solution. To this solution was added compound 21 (trihydrochloride salt, 0.30 g, 0.48 mmol) and pyridine (0.24 g, 3.0 mmol). The resultant mixture was stirred at room temperature for 12 h, and concentrated. The residue was purified by flash chromatography on silica gel (eluent EtOAc/MeOH) to afford title compound 75 (0.093 g, 29% yield) as a white solid. ¹H NMR (300 MHz, MeOH-d₄) δ (ppm): 8.62 (d, J=1.8Hz, 1H), 8.51 (d, J=5.7 Hz, 1H), 8.13 (d, J=7.8 Hz, 1H), 8.12 (s, 1H), 7.96 (dd, J=2.1, 8.1 Hz, 1H), 7.71 (dd, J=2.1, 12.6 Hz, 1H), 7.34 (t, J=8.7 Hz, 1H), 7.26-7.23 (m, 1H), 6.68 (dd, J=1.2, 5.4 Hz, 1H), 4.26-4.23 (m, 2H), 3.75-3.64 (m, 6H), 3.62-3.53 (m, 6H), 3.39 (s, 3H), 2.66 (sep, J=3.6 Hz, 1H), 2.58-2.50 (m, 4H), 0.84-0.76 (m, 2H), 0.60-0.54 (m, 2H) [Peaks of the two NH protons were not observed]. MS (m/z): 664.9 (M+H)⁺, 687.5 (M+Na)⁺.

### Example 46

### 2-Methoxyethyl 4-((6-(7-(4-(3-cyclopropylureido)-2-fluorophenoxy)thieno[3,2-b]pyridin-2-yl)pyridin-3-yl)methyl)piperazine-1-carboxylate (76)

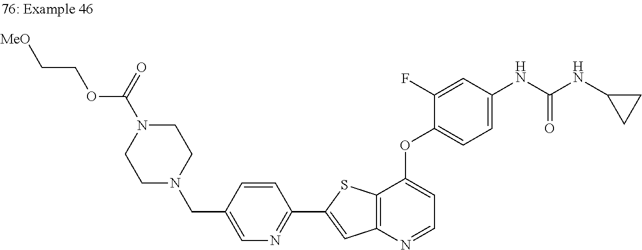 [SMILES: COCCOC(=O)N1CCN(Cc2ccc(-c3cc4nccc(Oc5ccc(NC(=O)NC6CC6)cc5F)c4s3)nc2)CC1]

Compound 76 (example 46) was prepared starting from 2-methoxyethanol and following the procedures similar to the ones described above for the synthesis of compound 75 (example 45, scheme 15). ¹H NMR (300 MHz, MeOH-d₄) δ (ppm): 8.62 (d, J=1.5Hz, 1H), 8.51 (d, J=5.7 Hz, 1H), 8.13(d, J=7.8 Hz, 1H), 8.12 (s, 1H), 7.96 (dd, J=2.1, 8.1 Hz, 1H), 7.71 (dd, J=2.1, 12.6 Hz, 1H), 7.34 (t, J=8.7 Hz, 1H), 7.26-7.23 (m, 1H), 6.68 (dd, J=1.2, 5.7 Hz, 1H), 4.26-4.23 (m, 2H), 3.69 (s, 2H), 3.66-3.64 (m, 2H), 3.60-3.52 (m, 4H), 3.41 (s, 3H), 2.66 (sep, J=3.3 Hz, 1H), 2.58-2.50 (m, 4H), 0.84-0.76 (m, 2H), 0.60-0.54 (m, 2H). [Peaks of the two NH protons were not observed]. MS (m/z): 621.0 (M+H)⁺, 643.3 (M+Na) ⁺.

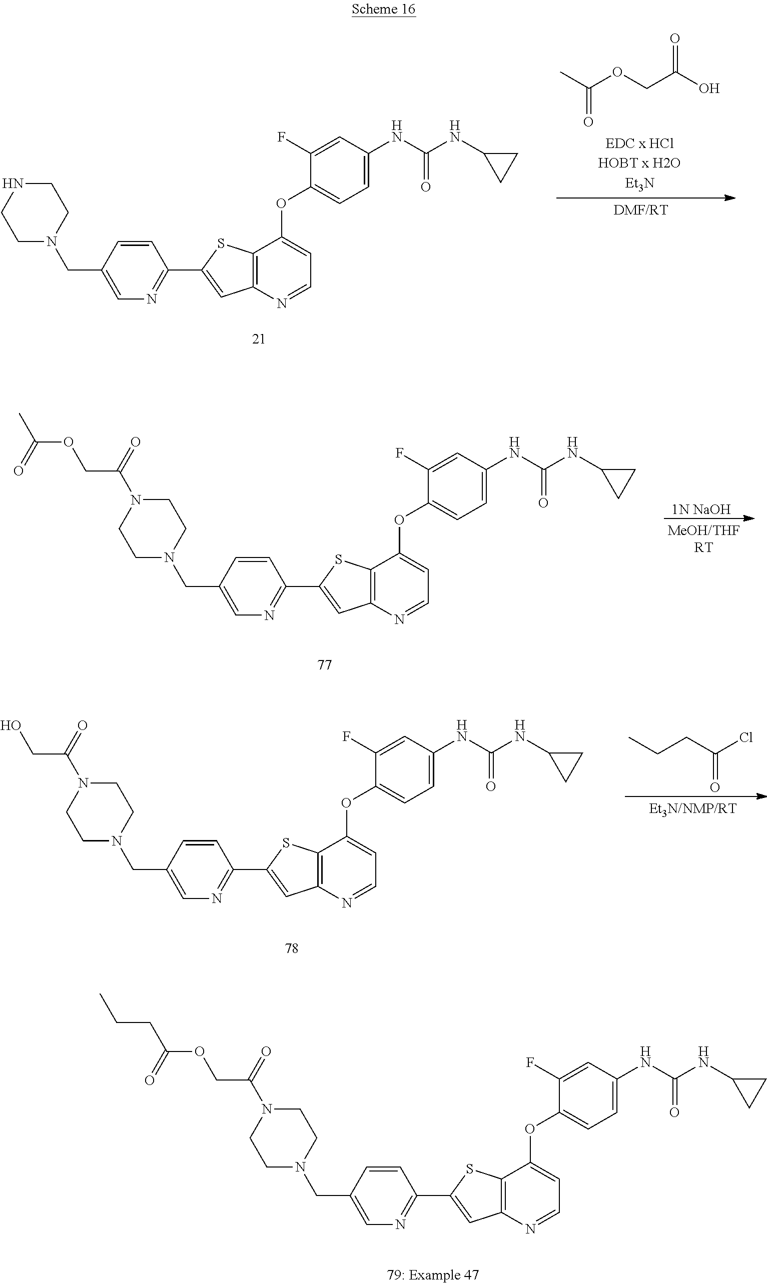

### Example 47

### 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl butyrate (79)

Step 1. 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl acetate

To a stirred solution of compound 21 (600 mg, 1.16 mmol, scheme 3), 2-acetoxyacetic acid (205 mg, 1.74 mmol) and tiethylamine (481 μl, 3.47 mmol) in DMF (15 ml) under nitrogen were added HOBT monohydrate (195 mg, 1.27 mmol) and EDC hydrochloride (444 mg, 2.31 mmol). The reaction mixture was stirred at rt overnight, quenched by addition of water, and diluted with AcOEt with traces of MeOH to form a biphasic system. The phases were separated; the organic layer was successively washed with a saturated aqueous solution of sodium bicarbonate and brine, dried over anhydrous magnesium sulfate, filtered and concentrated. The residue was purified by Biotage (Snap 50g cartridge; MeOH/DCM: 0/100 to 10/90 over 20 CV then 10/90 over 5 CV), to afford the desired product 77 (537 mg, 0.868 mmol, 75% yield) as an off-white sticky solid. MS (m/z): 647.1 (M+H).

Step 2. 1-Cyclopropyl-3-(3-fluoro-4-(2-(5((4-(2-hydroxyacetyl)piperazin-1-yl)methyl)pyridin-2-yl)thieno[3,2-b]pyridin-7-yloxy)phenyl)urea (78)

To a stirred solution of compound 77 (0.94 g, 1.52 mmol) in a mixture of MeOH/THF (30 ml/25 ml) was added 1N NaOH (3.8 ml, 3.80 mmol). The reaction mixture was stirred at rt for 3 h, concentrated, diluted in a minimum of methanol in water, neutralyzed with a saturated aqueous solution of ammonium chloride (pH around 8). The solid was collected by filtration, rinsed with water and dried to afford the desired product 78 (826 mg, 1.43 mmol, 94% yield) as an off-white fluffy solid. ¹H NMR (400 MHz, DMSO-d₆) δ (ppm): 8.77-8.69 (m, 1H), 8.57 (d, J=1.6 Hz, 1H), 8.52 (d, J=5.5 Hz, 1H), 8.34 (s, 1H), 8.26 (d, J=8.0 Hz, 1H), 7.88 (dd, J=8.1, 2.1 Hz, 1H), 7.73 (dd, J=13.5, 2.3 Hz, 1H), 7.38 (t, J=9.1 Hz, 1H), 7.20 (bd, J=9.2 Hz, 1H), 6.65 (d, J=4.9 Hz, 1H), 6.63-6.56 (m, 1H), 4.55 (t, J=5.5 Hz, 1H), 4.07 (d, J=5.5 Hz, 2H), 3.60 (s, 2H), 3.53-3.43 (m, 2H), 2H are hidden, 2.59-2.51 (m, 1H), 2.45-2.33 (m, 4H), 0.72-0.58 (m, 2H), 0.50-0.36 (m, 2H). MS (m/z): 577.5 (M+H).

Step 3. 2-(4-46-(7-(4-(3-Cyclopropylureido)-2-fluorophenoxy)thieno[3,2-b]pyridin-2-yl)pyridin-3-yl)methyl)piperazin-1-yl)-2-oxoethyl butrate

To a stirred solution of compound 78 (288 mg, 0.50 mmol) and Et₃N (151 mg, 3 eq) in NMP (2 mL) was added butyryl chloride (106 mg, 2 eq) at room temperature and the reaction mixture was stirred for 1 h. The reaction was quenched with water (10 mL) and extracted with CH₂Cl₂. The organic extract was dried over MgSO₄, concentrated under reduced pressure and the residue was purified by flash column chromatography (NH silica, Hexane/AcOEt=50/50-0/100) to afford title compound 79. ¹H NMR (300 MHz, CDCl₃) δ (ppm): 8.57 (s, 1H), 8.43 (d, J=4.8 Hz, 1H), 8.08-7.82 (m, 2H), 7.86-7.68 (m, 2H), 7.58 (d, J=12.0 Hz, 1H), 7.24-7.00 (m, 2H), 6.61-6.36 (m, 1H), 5.93-5.62 (m, 1H), 4.71 (s, 2H), 3.81-3.23 (m, 5H), 2.85-2.22 (m, 8H), 1.67 (q, J=6.9 Hz, 2H), 1.07-0.88 (m, 3H), 0.88-0.66 (m, 2H), 0.66-0.44 (m, 2H). MS (m/z): 647.1 (M+H)⁺.

### Example 48

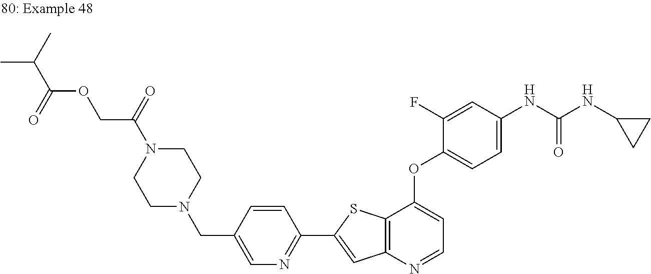 [SMILES: CC(C)C(=O)OCC(=O)N1CCN(Cc2ccc(-c3cc4nccc(Oc5ccc(NC(=O)NC6CC6)cc5F)c4s3)nc2)CC1]

### 2-(4-((6-(7-(4-(3-Cyclopropylureido)-2-fluorophenoxy)thieno[3,2-b]pyridin-2-yl)pyridin-3-yl)methul)piperazin 1-yl)-2-oxoethyl isobutrate (80)

Compound 80 (example 48) was prepared starting from compound 78 and following the procedures similar to the ones described above for the synthesis of compound 79 (example 47, scheme 16) and using isobutyryl chloride instead of butyryl chloride. ¹H NMR (300 MHz, CDCl₃) δ (ppm): 8.57 (s, 1H), 8.48 (d, J=4.8 Hz, 1H), 7.98 (s, 1H), 7.92-7.68 (m, 2H), 7.62 (d, J=12.0 Hz, 1H), 7.50-7.31 (m, 1H), 7.24-7.08 (m, 2H), 6.52 (d, J=4.8 Hz, 1H), 4.73 (s, 2H), 3.79-3.51 (m, 3H), 3.51-3.30 (m, 2H), 2.78-2.57 (m, 2H), 2.57-2.35 (m, 4H), 2.14-1.67 (m, 1H), 1.23 (d, J=6.6 Hz, 6H), 0.99-0.78 (m, 2H), 0.78-0.56 (m, 2H). MS (m/z): 647.3 (M+H)⁺.

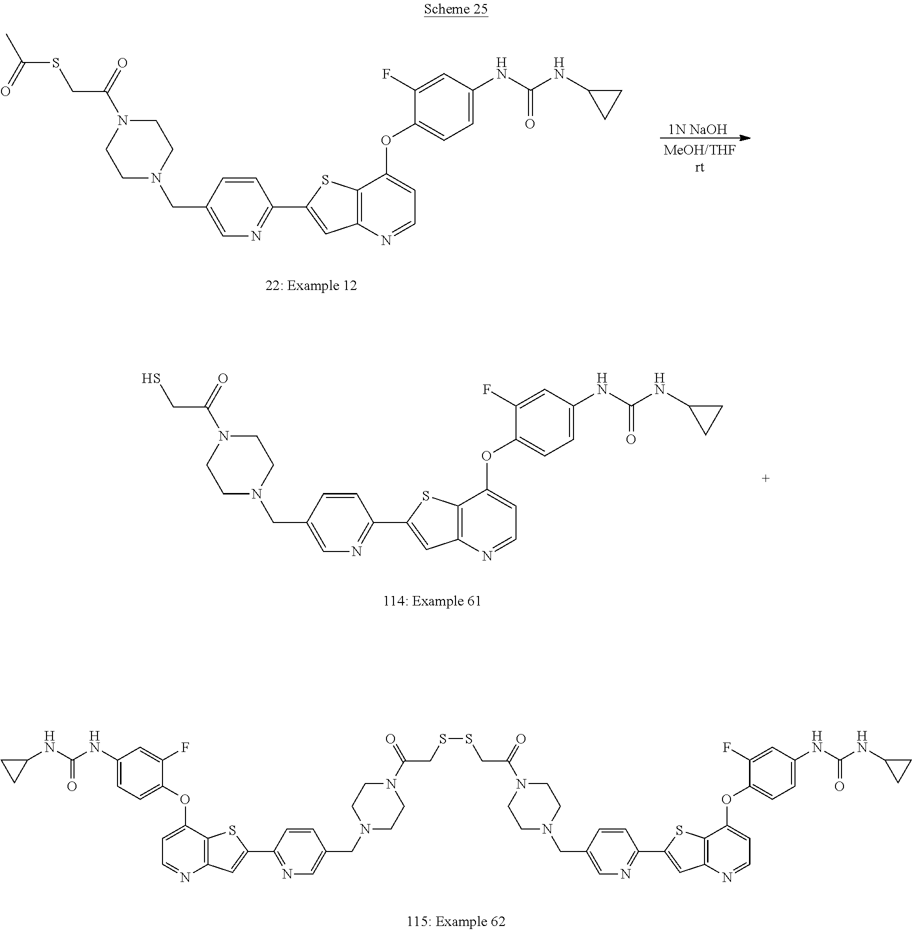

### Examples 61 and 62

### 1-Cyclopropyl-3-(3-fluoro-4-(2-(5-((4-(2-mercaptoacetyl)piperazin-1-yl)methyl)pyridin-2-yl)thieno[3,2-b]pyridin-7-yloxy)phenyl)urea (114) and 3-cyclopropyl-N-(4-(2-(5-((4-(2-((2-(4-((6-(7-(4-(3-cyclopropylureido)-2-fluorophenoxy)thieno[3,2-b]pyridin-2-yl)pyridin-3-yl)methyl)piperazin-1-yl)-2-oxoethyl)disulfanyl)acetyl)piperazin-1-yl)methyl)pyridin-2-yl)thieno[3,2-b]pyridin-7-yloxy)-3-fluorophenyl)urea (115)

To a stirred solution of compound 22 (115 mg, 0.18 mmol) in a mixture of MeOH/THF (5/5 ml) under nitrogen was added 1N NaOH (0.91 ml). The reaction mixture was stirred at room temperature for 1 h, concentrated, diluted with MeOH, and further with with water to form a precipitate that was sonicated for 15 min, collected by filtration, rinsed with water and dried under high vacuum. The dry material was purified by Biotage (Snap 25 g cartridge; MeOH/DCM: 1/99 to 10/90 over 30 CV, then 10/90 to 30/70 over 20 CV), to afford the thiol 114 (8.2 mg, 0.014 mmol, 7% yield) as white sticky solid and the disulfide 115 (40 mg, 0.034 mmol, 18%) as an off-white solid.

Characterization of 114: ¹H NMR (400 MHz, DMSO-d₆) δ (ppm): mixture of rotamers, 8.70 (s, 1H), 8.57 (bs, 1H), 8.52 (d, J=5.5 Hz, 1H), 8.34 (s, 1H), 8.26 (d, J=8.2 Hz, 1H), 7.88 (dd, J=8.2, 2.0 Hz, 1H), 7.73 (dd, J=13.7, 2.3 Hz, 1H), 7.38 (t, J=9.1 Hz, 1H), 7.20 (bd, J=9.0 Hz, 1H), 6.65 (d, J=4.9 Hz, 1H), 6.56 (bs, 1H), 3.60 (s, 2H), 3.55-3.36 (m, 6H), 2.59-2.52 (m, 1H), 2.48-2.32 (m, 4H), 0.72-0.58 (m, 2H), 0.50-0.36 (m, 2H), one SH is missing. MS (m/z): 593.2 (M+H).

Characterization of 115: ¹H NMR (400 MHz, DMSO-d₆) δ (ppm): 8.77 (s, 2H), 8.57 (bd, J=1.4 Hz, 2H), 8.51 (d, J=5.2 Hz, 2H), 8.33 (s, 2H), 8.25 (d, J=8.0 Hz, 2H), 7.87 (dd, J=8.2, 2.0 Hz, 2H), 7.73 (dd, J=13.6, 2.4 Hz, 2H), 7.37 (t, J=9.1 Hz, 2H), 7.19 (bd, J=8.8 Hz, 2H), 6.64 (d, J=5.1 Hz, 2H), 6.61 (bd, J=2.3 Hz, 2H), 3.84 (s, 4H), 3.59 (s, 4H), 3.54-3.42 (m, 8H), 2.59-2.52 (m, 2H), 2.48-2.32 (m, 8H), 0.72-0.58 (m, 4H), 0.49-0.36 (m, 4H). MS (m/z): 1183.7 (M+H).

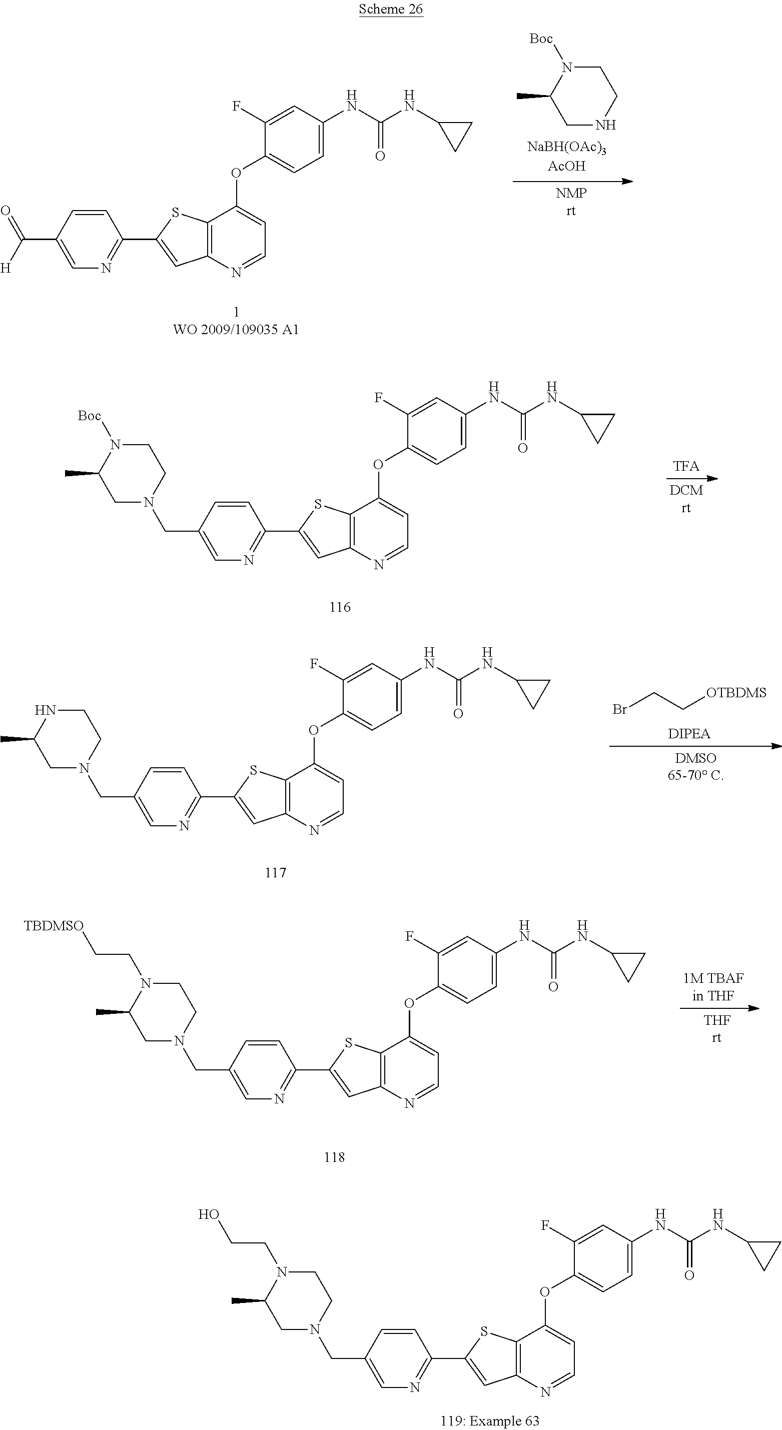

### Example 63

### (R)-1-Cyclopropyl-3-(3-fluoro -4-(2-(5-((4-(2-hydroxyethyl)-3-methylpip erazin-1-yl)methyl)pyridin-2-yl)thieno[3,2-b]pyridin-7-yloxy)phenyl)urea (119)

Step 1. (R)-tent-butyl 4-4-((6-(7-(4-(3-cyclopropylureido)-2-fluorophenoxy)thieno[3,2-b]pyridin-2-yl)pyridin-3-yl)methyl)-2-methylpiperazine-1-carboxylate (116)

To a solution of compound 1 (1.330 g, 2.97 mmol), (R)-1-N-Boc-2-methyl piperazine (713 mg, 3.56 mmol) in NMP (30 ml) and acetic acid (339 μl, 5.93 mmol) at rt under nitrogen was added portionwise NaBH(OAc)₃ (2.183 g, 9.79 mmol) over 2 hrs. The reaction mixture was stirred at rt overnight, poured into a stirred saturated aqueous solution of sodium bicarbonate, and stirred for 30 min. The precipitate was collected by filtration, rinsed with water and dried. The material was absorded on silica gel and purified twice by Biotage (Snap 50 g cartridge: 2% of ammonium hydroxyde in MeOH/DCM: 1/99 to 10/90 over 30 CV), to afford the desired product 116 (922 mg, 1.45 mmol, 49% yield) as a beige sticky solid. MS (m/z): 633.38 [M+H].

Step 2. (R)-1-cyclopropyl-3-(3-fluoro-4-(2-(5-((3-methylpiperazin-1-yl)methyl)pyridin-2-yl)thieno[3,2-b]pyridin-7-yloxy)phenyl)urea (117)

A solution of compound 116 (922 mg, 1.457 mmol) and TFA (5.61 ml) in DCM (30 ml) was stirred at rt for 2.5 h. The reaction mixture was concentrated (azeotropes with DCM), diluted with water (with traces of methanol), and poured into a mixture of saturated aqueous solution of sodium bicarbonate and 1N NaOH to form a precipitate that was shaken for 30 min, collected by filtration, rinsed with water and dried. The material was dissolved with DCM/methanol, dried over magnesium sulfate, filtered, concentrated and dried under high vacuum to afford the desired product 117 (748 mg, 1.40 mmol, 96% yield) as a beige-pale brown solid. MS (m/z): 533.46 [M+H].

Step 3. (R)-1-4-(2-(5-((4-(2-(tert-butyldimethylsilyloxy)ethyl)-3-methylpiperazin-1-yl)methyl)pyridin-2-yl)thieno[3,2-b]pyridin-7-yloxy)-3-fluorophenyl)-3-cyclopropylurea (118)

To a stirred solution of compound 117 (200 mg, 0.375 mmol) in DMSO (5 ml) under nitrogen were added DIPEA (197 μl, 1.13 mmol) and (2-bromoethoxy)-tert-butyldimethylsilane (403 μl, 1.88 mmol), and the reaction mixture was heated at 65-70° C. for 4.5 h. The reaction mixture was diluted with AcOEt and successively washed with a saturated aqueous solution of sodium bicarbonate, a saturated aqueous solution of ammonium chloride, water and brine, dried over anhydrous magnesium sulfate, filtered and concentrated. The residue was purified by Biotage (Snap 25 g cartridge; MeOH/DCM: 1/99 to 10/90 over 30 CV), to afford the desired product 118 (164 mg, 0.237 mmol, 63% yield) as a pale green sticky solid. MS (m/z): 691.5 [M+H].

Step 4. (R)-1-cyclopropyl-3-(3-fluoro-4-(2-(5-((4-(2-hydroxyethyl)-3-methylpiperazin-1-yl)methyl)pyridin-2-yl)thieno[3,2-b]pyridin-7-yloxy)phenyl)urea (119)

To a stirred solution of compound 118 (164 mg, 0.237 mmol) in THF (10 ml) at rt was added a solution of TBAF (1.9 mL, 1.9 mmol). The reaction mixture was stirred at rt for 2.5 hrs, and treated with more TBAF (2 mL, 2 mmol). The stirring was continued for another 1.5 hrs at rt, the reaction mixture was concentrated, diluted with water, neutralyzed with a saturated aqueous solution of sodium bicarbonate to form a precipitate. The precipitate was collected by filtration, rinsed with water and dried. The crude product was purified by Biotage (Snap 25 g cartridge, 2% of ammonium hydroxide in MeOH/DCM: 1/99 to 20/80 over 30 CV), to afford the desired product 119 (103 mg, 0.18 mmol, 75% yield) as an off-white sticky solid. ¹H NMR (400 MHz, DMSO-d₆) δ (ppm): 8.71 (s, 1H), 8.54 (bd, J=1.8 Hz, 1H), 8.52 (d, J=5.5 Hz, 1H), 8.32 (s, 1H), 8.24 (d, J=8.2 Hz, 1H), 7.85 (dd, J=8.2, 2.0 Hz, 1H), 7.73 (dd, J=13.6, 2.4 Hz, 1H), 7.38 (t, J=9.1 Hz, 1H), 7.20 (bd, J=10.2 Hz, 1H), 6.64 (d, J=5.3 Hz, 1H), 6.57 (bd, J=2.5 Hz, 1H), 4.34 (t, J=5.4 Hz, 1H), 3.51 (s, 2H), 3.49-3.40 (m, 2H), 2.82-2.52 (m, 5H), 2.47-2.10 (m, 4H), 1.94-1.81 (m, 1H), 0.95 (d, J=6.3 Hz, 3H), 0.72-0.58 (m, 2H), 0.50-0.36 (m, 2H). MS (m/z): 577.50 [M+H].

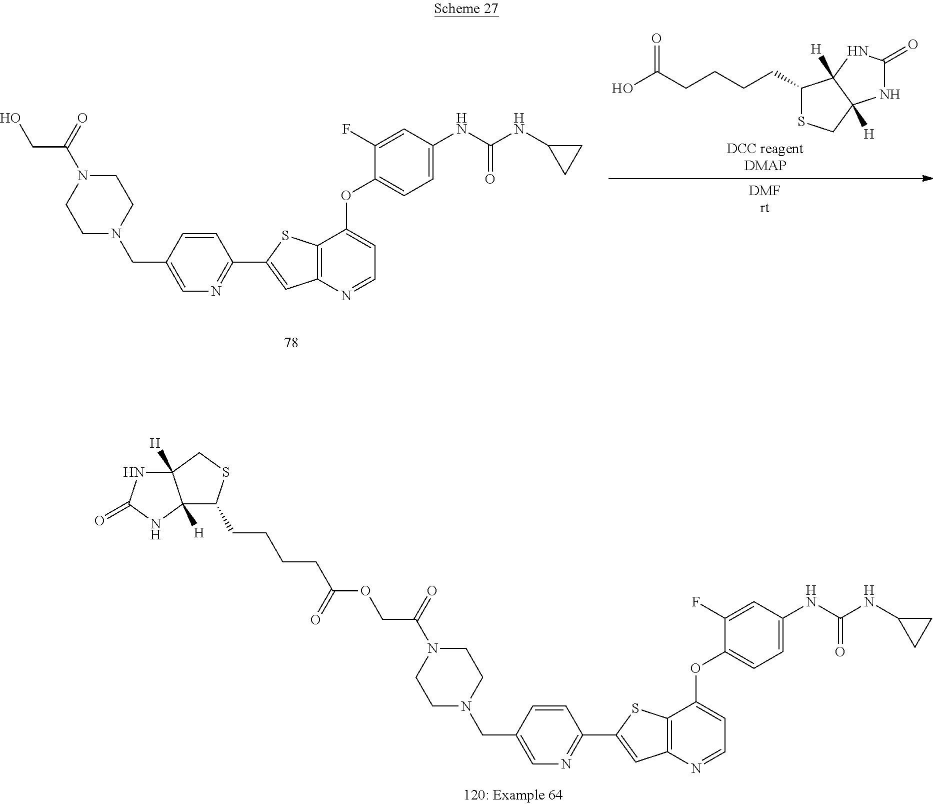 [SMILES: O=C(Nc1ccc(Oc2ccnc3cc(-c4ccc(CN5CCN(C(=O)CO)CC5)cn4)sc23)c(F)c1)NC1CC1 ; O=C(O)CCCC[C@H]1SC[C@H]2NC(=O)N[C@H]21 ; O=C(Nc1ccc(Oc2ccnc3cc(-c4ccc(CN5CCN(C(=O)COC(=O)CCCC[C@H]6SC[C@H]7NC(=O)N[C@H]76)CC5)cn4)sc23)c(F)c1)NC1CC1]

### Example 64

### 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl 5-((3aR,4R,6aS)-2-oxohexahydro-1H-thieno[3,4-d]imidazol-4-yl)pentanoate (120)

To a stirred solution of 78 (150 mg, 0.26 mmol, scheme 16), D-biotin (159 mg, 0.65 mmol) and DMAP (33 mg, 0.27 mmol) in DMF (10 ml) under nitrogen was added DCC (215 mg, 1.04 mmol), and the reaction mixture was stirred at rt overnight. The reaction mixture was partitioned between AcOEt and water. After separation, the organic layer was collected, successively washed with water and brine. A sticky solid precipitated on the walls of the separating funnel; the solid was dissolved in methanol/DCM mixture and combined with the organic phase. The combined organic phase was concentrated and the residue was purified by Biotage (Snap 25 g cartridge; MeOH/DCM: 1/99 to 20/80 over 30 CV), to afford the desired product 120 (80 mg, 0.10 mmol, 38% yield) as an off-white sticky solid. ¹H NMR (400 MHz, DMSO-d₆) δ (ppm): 8.74 (s, 1H), 8.58 (bd, J=1.6 Hz, 1H), 8.52 (d, J=5.5 Hz, 1H), 8.34 (s, 1H), 8.26 (d, J=8.0 Hz, 1H), 7.88 (dd, J=8.1, 2.1 Hz, 1H), 7.73 (dd, J=13.5, 2.3 Hz, 1H), 7.38 (t, J=9.1 Hz, 1H), 7.20 (bd, J=9.8 Hz, 1H), 6.65 (d, J=5.3 Hz, 1H), 6.60 (bd, J=2.5 Hz, 1H), 6.43 (s, 1H), 6.36 (s, 1H), 4.78 (s, 2H), 4.33-4.26 (m, 1H), 4.16-4.10 (m, 1H), 3.60 (s, 2H), 3.52-3.34 (m, 4H), 3.13-3.05 (m, 1H), 2.82 (dd, J=12.4, 5.0 Hz, 1H), 2.61-2.52 (m, 2H), 2.47-2.30 (m, 6H), 1.68-1.28 (m, 6H), 0.72-0.58 (m, 2H), 0.50-0.36 (m, 2H). MS (m/z): 803.52 [M+H].

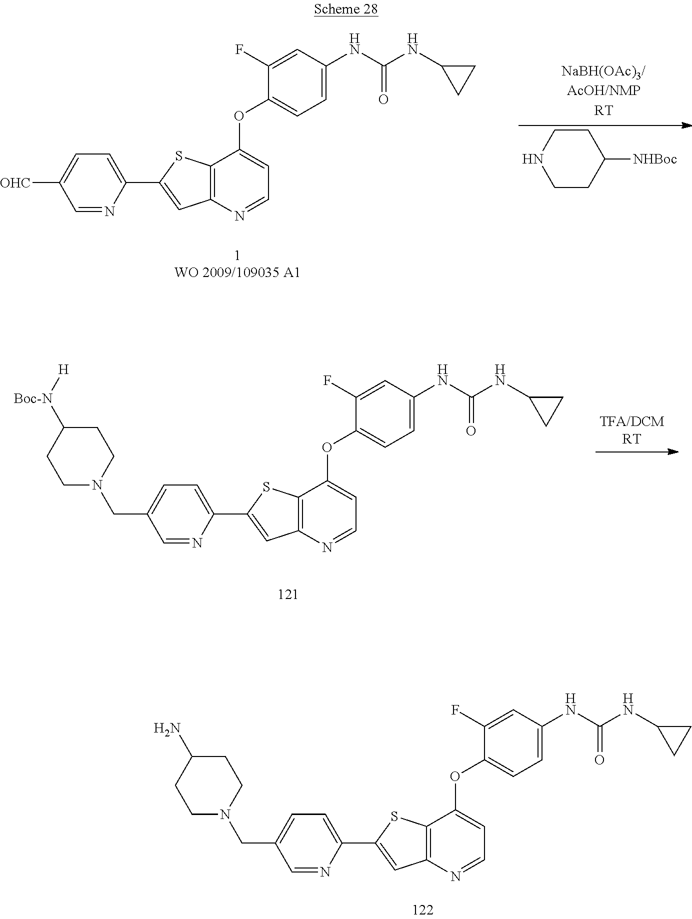

### 1-(4-(2-(5-((4-Aminopiperidin-1-yl)methyl)pyridin-2-yl)thieno[3,2-b]pyridin-7-yloxy)-3-fluorophenyl)-3-cyclopropylurea (122)

Step 1. tert-Butyl 1-((6-(7-(4-(3-cyclopropylureido)-2-fluorophenoxy)thieno[3,2-b]pyridin-2-yl)pyridin-3-yl)methyl)piperidin-4-ylcarbamate (121).

tert-Butyl piperidin-4-ylcarbamate (1.34 g, 6.69 mmol) was added to a solution of aldehyde 1 (2.0 g, 4. 46 mmol) and glacial AcOH (0.250 mL) in NMP (20 mL). The reaction mixture was stirred for 30 min. NaBH(OAc)₃ was then added and the reaction mixture was stirred for an additional 2.5 hours. The reaction mixture was then poured into a saturated aqueous NaHCO₃ solution to form a precipitate that was collected by filtration, washed with water and dried. The crude material was purified by column chromatography using a 5 to 20% gradient of MeOH in EtOAc as eluent to afford the title compound 121 (1.45 g, 51.4% yield). MS (m/z): 633.6 (M+1)+

Step 2. 1-(4-(2-(5-((4-Aminopiperidin-1-yl)methyl)pyridin-2-yl)thieno[3,2-b]pyridin-7-yloxv)-3-fluorophenyl)-3-cyclopropylurea (122).

A solution of compound 121 in TFA (25 mL) was stirred at RT for 1.5 hours then evaporated. To the residue was added 3N aqueous NaOH solution and the suspension was stirred at RT overnight, collected by filtration, washed with water and dried to afford the title compound 122 (1.177 g, 96% yield). ¹H NMR (400 MHz, DMSO-d₆) δ (ppm): 8.75 (s, 1H); 8.53-8.51 (m, 2H); 8.32 (s, 1H); 8.23 (d, J=8.2 Hz, 1H); 7.84 (dd, J=8.2, 2.2 Hz, 1H); 7.73 (dd, J=13.5, 2.3 Hz, 1H); 7.38 (t, J=9.0 Hz, 1H); 7.20 (dd, J=8.8 1.2 Hz, 1H); 6.64 (d, J=5.5 Hz 1H); 6.61 (d, J=2.3 Hz, 1H); 3.52 (s, 2H); 2.74 (d, J=11.3 Hz, 2H); 2.58-2.52 (m, 1H); 1.99 (t, J=9.8 Hz, 2H); 1.66 (d, J=11.3 Hz, 2H); 1.29-1.20 (m, 2H); 0.68-0.63 (m, 2H); 0.45-0.41 (m, 2H). [Signal of the NH₂-group is not seen; NH₂—CH-signal is obscured by the peak of residual water]. MS (m/z): 533.5 (M+1)+

### Pharmaceutical Compositions

In some embodiments, the invention provides pharmaceutical compositions comprising a compound according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In some embodiments, compositions of the invention are administered intravenously in a hospital setting. In some embodiments, administration may be by the oral route.

The characteristics of the carrier, excipient or diluent will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

The active compound is included in the pharmaceutically acceptable carrier, excipient or diluent in an amount sufficient to deliver to a patient a therapeutically effective amount without causing serious toxic effects in the patient treated. The effective dosage range of a pharmaceutically acceptable derivative can be calculated based on the weight of the parent compound to be delivered. If the derivative exhibits activity in itself, the effective dosage can be estimated as above using the weight of the derivative, or by other means known to those skilled in the art.

### Inhibition of VEGF Receptor Signaling

In some embodiments the invention provides a method of inhibiting VEGF receptor signaling in a cell, comprising contacting a cell in which inhibition of VEGF receptor signaling is desired with an inhibitor of VEGF receptor signaling according to the invention. Because compounds of the invention inhibit VEGF receptor signaling, they are useful research tools for in vitro study of the role of VEGF receptor signaling in biological processes. In some embodiments, inhibiting VEGF receptor signaling causes an inhibition of cell proliferation of the contacted cells.

### ASSAY EXAMPLES

### Inhibition of VEGF Activity

The following protocol was used to assay the compounds of the invention.

### Assay Example 1

In Vitro Receptor Tyrosine Kinase Assay (VEGF receptor KDR)

This test measures the ability of compounds to inhibit the enzymatic activity of recombinant human VEGF receptor enzymatic activity.

A 1.6-kb cDNA corresponding to the catalytic domain of VEGFR2 (KDR) (Genbank accession number AF035121 amino acid 806 to 1356) is cloned into the Pst I site of the pDEST20 Gateway vector (Invitrogen) for the production of a GST-tagged version of that enzyme. This construct is used to generate recombinant baculovirus using the Bac-to-Bac™ system according to the manufacturer's instructions (Invitrogen).

The GST-VEGFR2806-1356 protein is expressed in Sf9 cells (Spodoptera frugiperda) upon infection with recombinant baculovirus construct. Briefly, Sf9 cells grown in suspension and maintained in serum-free medium (Sf900 II supplemented with gentamycin) at a cell density of about 2×106 cells/ml are infected with the above-mentioned viruses at a multiplicity of infection (MOI) of 0.1 during 72 hours at 27° C. with agitation at 120 rpm on a rotary shaker. Infected cells are harvested by centrifugation at 398g for 15 min. Cell pellets are frozen at −80° C. until purification is performed.

All steps described in cell extraction and purification are performed at 4° C. Frozen Sf9 cell pellets infected with the GST-VEGFR2806-1356 recombinant baculovirus are thawed and gently resuspended in Buffer A (PBS pH 7.3 supplemented with 1 μg/ml pepstatin, 2 μg/ml Aprotinin and leupeptin, 50 μg/ml PMSF, 50 μg/ml TLCK and 10 μM E64 and 0.5 mM DTT) using 3 ml of buffer per gram of cells. Suspension is Dounce homogenized and 1% Triton X-100 is added to the homogenate after which it is centrifuged at 22500 g, 30 min., 4oC. The supernatant (cell extract) is used as starting material for purification of GST-VEGFR2806-1356.

The supernatant is loaded onto a GST-agarose column (Sigma) equilibrated with PBS pH 7.3. Following a four column volume (CV) wash with PBS pH 7.3+1% Triton X-100 and 4 CV wash with buffer B (50mM Tris pH 8.0, 20% glycerol and 100mM NaCl), bound proteins are step eluted with 5 CV of buffer B supplemented with 5 mM DTT and 15 mM glutathion. GST-VEGFR2806-1356 enriched fractions from this chromatography step are pooled based on U.V. trace i.e. fractions with high 0.D.280. Final GST-VEGFR2806-1356 protein preparations concentrations are about 0.7 mg/ml with purity approximating 70%. Purified GST-VEGFR2806-1356 protein stocks are aliquoted and frozen at −80° C. prior to use in enzymatic assay.

Inhibition of VEGFR/KDR is measured in a DELFIATM assay (Perkin Elmer). The substrate poly(Glu4, Tyr) is immobilized onto black high-binding polystyrene 96-well plates. The coated plates are washed and stored at 4° C. During the assay, the enzyme is pre-incubated with inhibitor and Mg-ATP on ice in polypropylene 96-well plates for 4 minutes, and then transferred to the coated plates. The subsequent kinase reaction takes place at 30° C. for 10-30 minutes. ATP concentrations in the assay are 0.6 uM for VEGFR/KDR (2× the Km). Enzyme concentration is 5 nM. After incubation, the kinase reactions are quenched with EDTA and the plates are washed. Phosphorylated product is detected by incubation with Europium-labeled anti-phosphotyrosine MoAb. After washing the plates, bound MoAb is detected by time-resolved fluorescence in a Gemini SpectraMax reader (Molecular Devices). Compounds are evaluated over a range of concentrations, and IC₅₀ values (concentration of compounds giving 50% inhibition of enzymatic activity) are determined. The results are shown in Table 9.

TABLE 9

| Cmpd # | VEGFR_IC50_UM |
|---|---|
| 31 | 0.017 |
| 34 | 0.005 |
| 35 | 0.004 |
| 36 | 0.004 |
| 35-A | 0.001 |
| 22 | 0.015 |
| 26 | 0.004 |
| 10 | 0.007 |
| 28 | 0.005 |
| 11 | 0.007 |
| 12 | 0.006 |
| 13 | 0.008 |
| 4 | 0.008 |
| 5 | 0.008 |
| 29 | 0.005 |
| 6 | 0.007 |
| 7 | 0.007 |
| 8 | 0.003 |
| 9 | 0.011 |
| 30 | 0.004 |
| 32 | 0.006 |
| 33 | 0.007 |
| 75 | 0.016 |
| 76 | 0.008 |
| 79 | 0.019 |
| 80 | 0.02 |
| 115 | |
| 114 | 0.01 |
| 119 | 0.008 |
| 120 | 0.021 |

### Assay Example 2

In vivo Choroidal Neovascularization (CNV) Model

This test measures the capacity of compounds to inhibit CNV progression. CNV is the main cause of severe vision loss in patients suffering from age-related macular degeneration (AMD).

Male Brown-Norway rats (Charles River Japan Co., Ltd.) were used in these studies.

Rats were anesthetized by intraperitoneal injection of pentobarbital, and the right pupil was dilated with 0.5% tropicamide and 0.5% phenylephrine hydrochloride. The right eye received 6 laser burns between retinal vessels using a slit lamp delivery system of Green laser Photocoagulator (Nidex Inc., Japan), and microscope slide glass with 10 mg/mL hyaluronic acid (SIGMA) used as a contact lens. The laser power was 200 mW for 0.1 second and spot diameter was 100 μm. At the time of laser burn, bubble production was observed; which is an indication of rupture of Bruch's membrane which is important for CNV generation.

After animals were anesthetized, and the right pupil dilated (as above mentioned), the right eye of the animal received the compound or vehicle by an injection (3 μL/eye) at doses of 3 or 10 nmol/eye on Day3. The compounds were dissolved or suspended in CBS, PBS, or other adequate vehicles before injection.

On Day 10, the animals were anesthetized with ether, and high molecular weight fluorescein isothiocyanate (FITC)-dextran (SIGMA, 2×106 MW) was injected via a tail vein (20 mg/rat). About 30 min after FITC-dextran injection, animals were euthanized by ether or carbon dioxide, and the eyes were removed and fixed with 10% formaline neutral buffer solution. After over 1 hour of fixation, RPE-choroid-sclera flat mounts were obtained by removing cornea, lens and retina from the eyeballs. The flat mounts were mounted in 50% glycerol on a microscope slide, and the portion burned by laser was photographed using a fluorescence microscope (Nikon Corporation, excitation filter: 465-495 nm, absorption filter: 515-555 nm). The CNV area was obtained by measurement of hyper-fluorescence area observed on the photograph using Scion image.

The average CNV area of 6 burns was used as an individual value of CNV area, and the average CNV area of compound treated group was compared with that of the vehicle-treated group. Results with some compounds of the present invention are shown in Table 10.

TABLE 10

| | Dose | Inhibition of CNV |
|---|---|---|
| Cmpd No. | (nmol/eye) | (%) |
| 31 | 10 | 74.9 |
| 34 | 3 | 72.1 |
| 35-A | 3 | 74.2 |
| 26 | 3 | 64.8 |
| 9 | 3 | 21.1 |
| 28 | 3 | 20.4 |
| 10 | 3 | 20.3 |
| 12 | 3 | 57.8 |
| 13 | 3 | 34.4 |
| 29 | 3 | 44.7 |
| 8 | 3 | 26.9 |
| 30 | 3 | 82.0 |
| 32 | 3 | 35.2 |
| 33 | 3 | 26.5 |
| 75 | 3 | 20.8 |
| 79 | 3 | 18.1 |
| 80 | 3 | 35.5 |

### Assay Example 3

### VEGF-dependent Erk Phosphorylation

Cells and growth factor: HUVEC cells are purchased from Cambrex Bio Science Walkersville, Inc and cultured according to the vendor's instructions. The full-length coding sequence of VEGF₁₆₅ is cloned using the Gateway Cloning Technology (Invitrogen) for baculovirus expression Sf9 cells. VEGF₁₆₅ is purified from conditioned media using a NaCl gradient elution from a HiTrap heparin column (GE Healthcare Life Sciences) followed by an imidazole gradient elution from a HiTrap chelating column (GE Healthcare Life Sciences), then buffer stored in PBS supplemented with 0.1% BSA and filter sterilized.

Cell assays: Cells are seeded at 8000 cells/ well of a 96 wells plate and grown for 48 hours. Cells are then grown overnight in serum and growth factor-free medium and exposed for 1.5 h to compounds dilutions. Following a 15 min incubation in medium, VEGF₁₆₅ (150 ng/ml) cells are lysed in ice-cold lysis buffer (50 mM HEPES, pH 7.4, 150 mM NaC1, 1.5 mM MgCl₂, 1% Triton X-100, 10% glycerol) containing 1 mM 4-(2 aminoethyl)benzenesulfonyl fluoride hydrochloride, 200 μM sodium orthovanadate, 1 mM sodium fluoride, 10 μg/mL leupeptin, 10 μg/mL aprotinin, 1 μg/mL pepstatin and 50 μg/mL Na-p-tosyl-L-lysine chloromethyl ketone hydrochloride and processed as Western blots to detect anti-phospho ERK1/2 (T202/Y204)(Cell Signaling Technologies).

Western blot analysis: lysates samples from single treatment wells are separated on 5-20% SDS-PAGE gels and immunobloting is performed using Immobilon polyvinylidene difluoride membranes (Amersham) according to the manufacturer's instructions. The blots are washed in Tris-buffered saline with 0.1% Tween 20 detergent (TBST) and probed for antibodies against phospho-Thr202/Tyr204-ERK (Cell signaling technologies. Chemiluminescence detection (Amersham, ECL plus) is performed according to the manufacturer's instructions using a Storm densitometer (GE Healthcare; 800 PMT, 100 nM resolution) for imaging and densitometry analysis. Values of over the range of dilution are used to prepare IC₅₀ curves using a 4-parameter fit model. These curves are calculated using GraFit 5.0 software.

### Assay Example 4

### In Vivo Solid Tumor Disease Model

This test measures the capacity of compounds to inhibit solid tumor growth.

Tumor xenografts are established in the flank of female athymic CD1 mice (Charles River Inc.), by subcutaneous injection of 1×106 U87, A431 or SKLMS cells/mouse. Once established, tumors are then serially passaged s.c. in nude mice hosts. Tumor fragments from these host animals are used in subsequent compound evaluation experiments. For compound evaluation experiments female nude mice weighing approximately 20 g are implanted s.c. by surgical implantation with tumor fragments of ˜30 mg from donor tumors. When the tumors are approximately 100 mm3 in size (˜7-10 days following implantation), the animals are randomized and separated into treatment and control groups. Each group contains 6-8 tumor-bearing mice, each of which is ear-tagged and followed individually throughout the experiment.

Mice are weighed and tumor measurements are taken by calipers three times weekly, starting on Day 1. These tumor measurements are converted to tumor volume by the well-known formula (L+W/4)3 4/3π. The experiment is terminated when the control tumors reach a size of approximately 1500 mm³. In this model, the change in mean tumor volume for a compound treated group / the change in mean tumor volume of the control group (non-treated or vehicle treated)×100 (ΔT/ΔC) is subtracted from 100 to give the percent tumor growth inhibition (% TGI) for each test compound. In addition to tumor volumes, body weight of animals is monitored twice weekly for up to 3 weeks.

### Assay Example 5

### VEGF-Induced Retinal Vascular Permeability in Rabbits

### Materials and Methods

This test measures the capacity of compounds to inhibit VEGF-induced retinal vascular permeability. Vascular permeability is the cause of severe vision loss in patients suffering from age-related macular degeneration (AMD). Female Dutch rabbits (˜2 kg; Kitayama LABES CO., LTD, Nagano, Japan) are anesthetized with pentobarbital and topically with 0.4% oxybuprocaine hydrochloride. Test articles or vehicle are injected into vitreous cavity after the dilation of the pupils with 0.5% tropicamide eye drop. Recombinant human VEGF₁₆₅ (500 ng; Sigma-Aldrich Co., St Louis, Mo.) is injected intravitreously 48 hr prior to the mesurement of vitreous fluorescein concentration. Rabbits are anesthetized with pentobarbital and sequentially injected sodium fluorescein (2 mg/kg) via the ear vein. Pupils are dilated with 0.5% tropicamide eye drop, and ocular fluorescein levels are measured using the FM-2 Fluorotron Master (Ocumetrics, Mountain View, Calif.) 30 min after fluorescein injection. The fluorescein concentrations in vitreous are obtained at data points that are 0.25 mm apart from posterior-end along an optical axis. Vitreous fluorescence concentration is considered fluorescein leakage from retinal vasculature. The average fluorescence peaks of the test article treated groups are compared with that of the vehicle-treated group.

### Assay Example 6

### Solubility Measurements.

The solubility of each substance was assessed using MultiScreen® HTS 96-well filtration system (filter; polycarbonate, pore size; 0.4 μm, Millipore). DMSO stock solutions of each test substance (10 mM) were prepared to initiate the assay. The equilibration was performed in PBS (pH 7.4) containing 100 μM of a test substance and 1% DMSO, for 24 hours at room temperature with shaking The concentrations of test substances in each filtrate were determined by HPLC-UV. The results with some compounds of the present invention are shown in Table 11.

HPLC conditions were following:

Waters ACQUITY UPLC H class instrument.

Column: Cadenza CD-C18 5 um 4.6×150 mm

Eluent A: 10 mM aqueous Ammonium formate

Eluent B: 0.1 volume % formic acid in acetonitrile

Flow: 1 mL/min, UV: 316 nm

0-2min: A/B=95/5

2-15min: A/B=95/5-30/70

15-20min: A/B=30/70

7(1-75min: A/B=95/5

TABLE 11

| Structure | Compd No | Solubility (μM) |
|---|---|---|
| 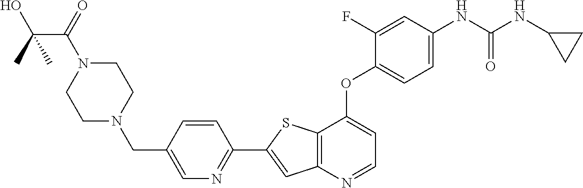 | 35-A | 1.19 |
| 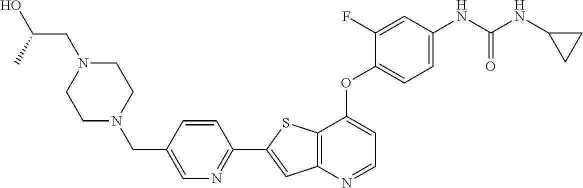 [SMILES: C[C@H](O)CN1CCN(Cc2ccc(-c3cc4nccc(Oc5ccc(NC(=O)NC6CC6)cc5F)c4s3)nc2)CC1] | 30 | 6.46 |

Table 11 reveals that the compounds of the present invention show good solubility.

What is claimed is:

1. A compound selected from the group consisting of 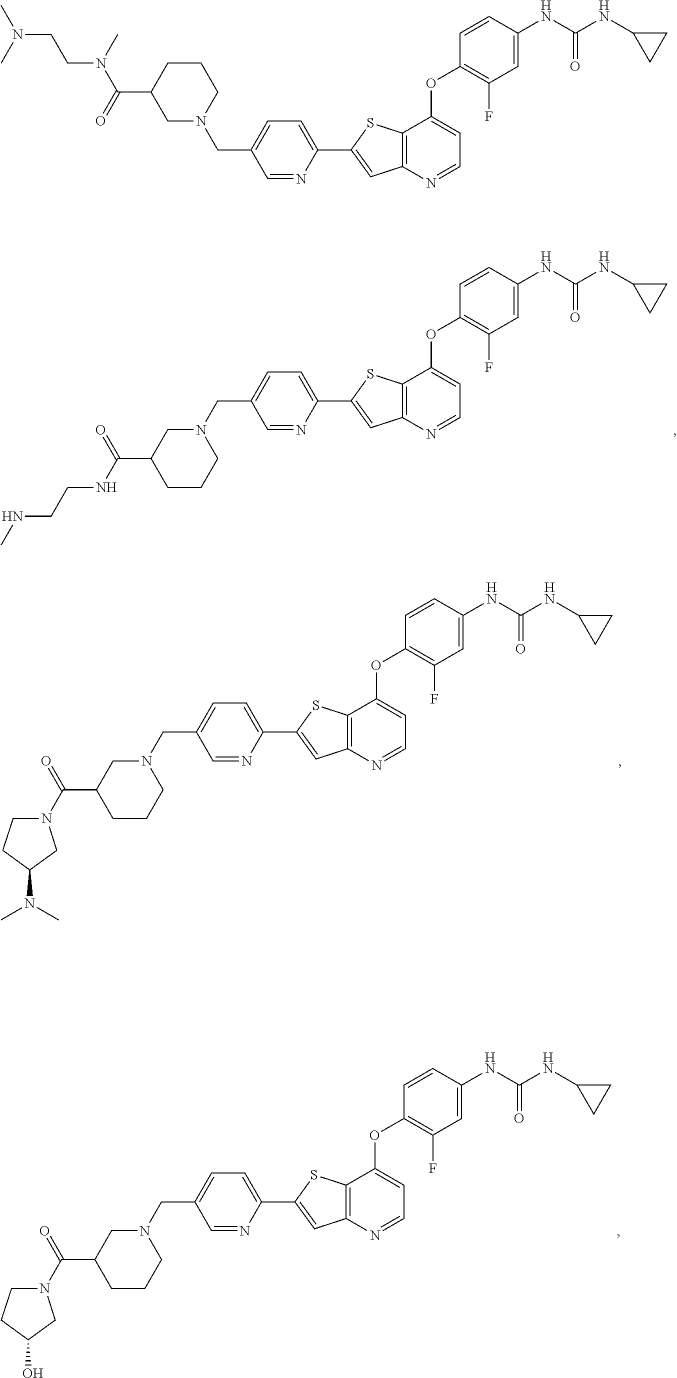 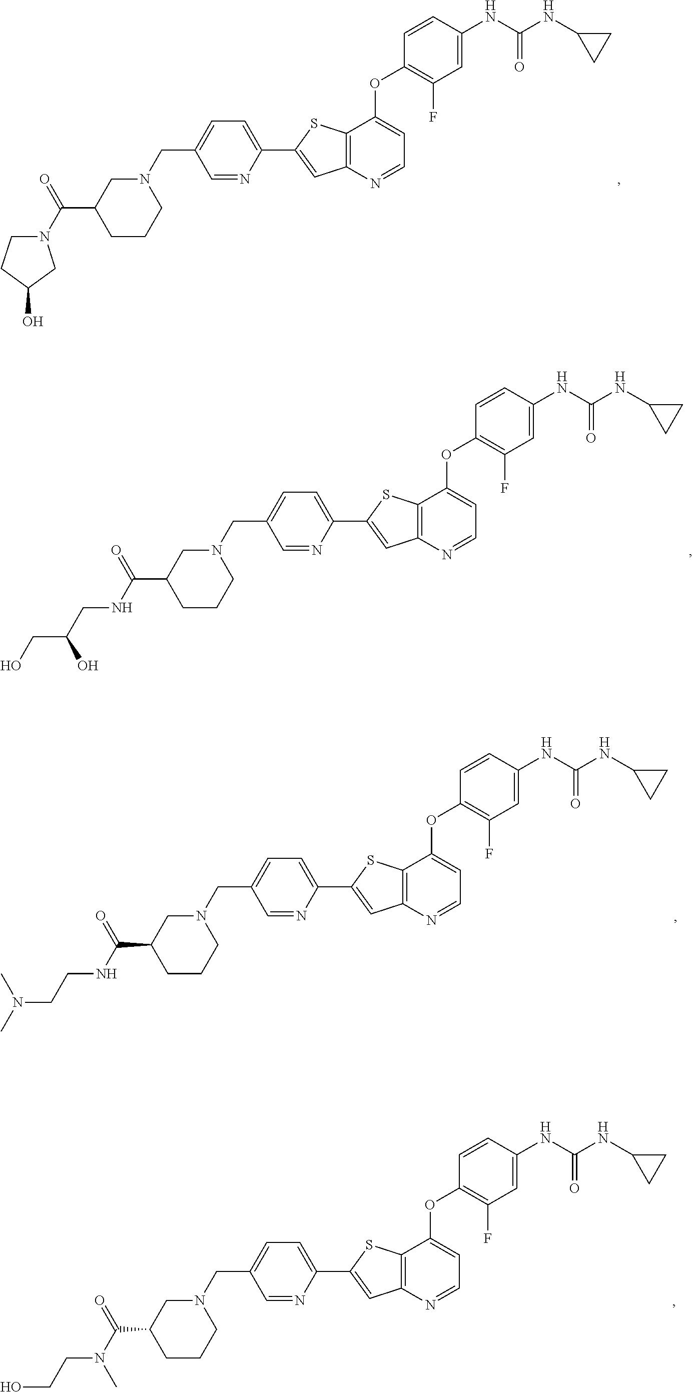 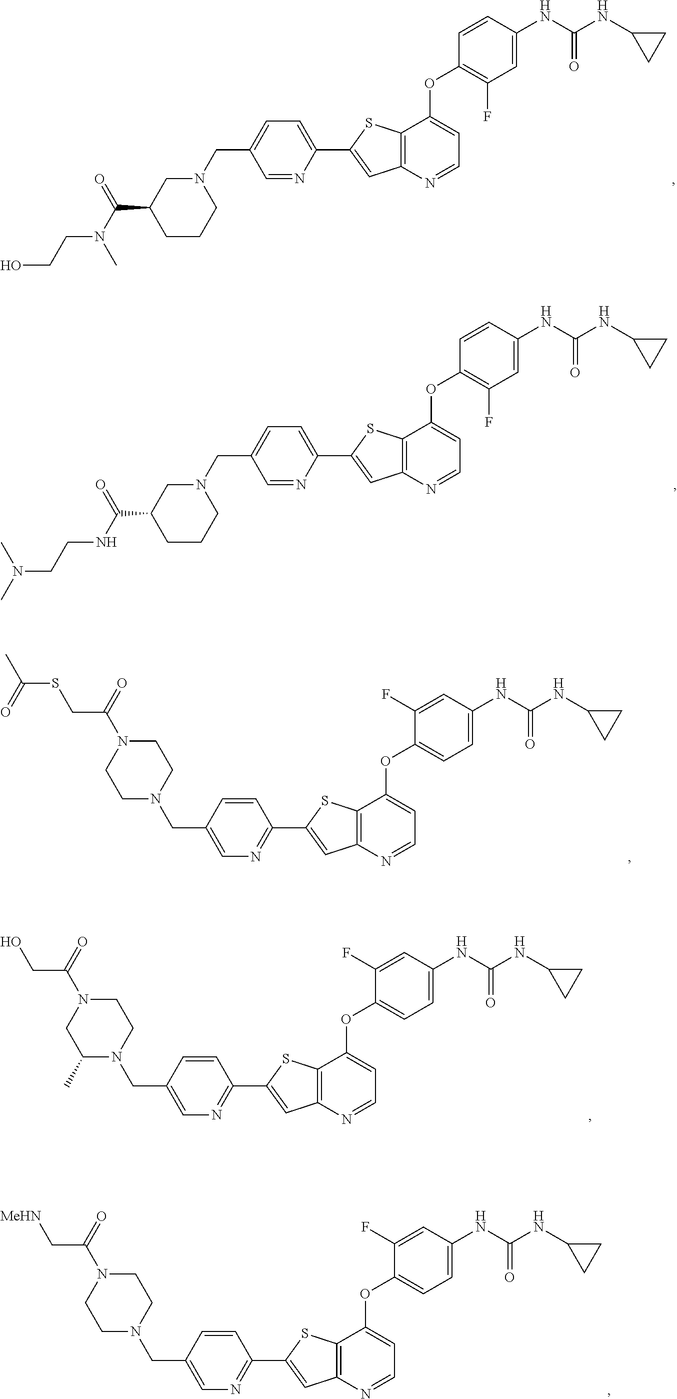 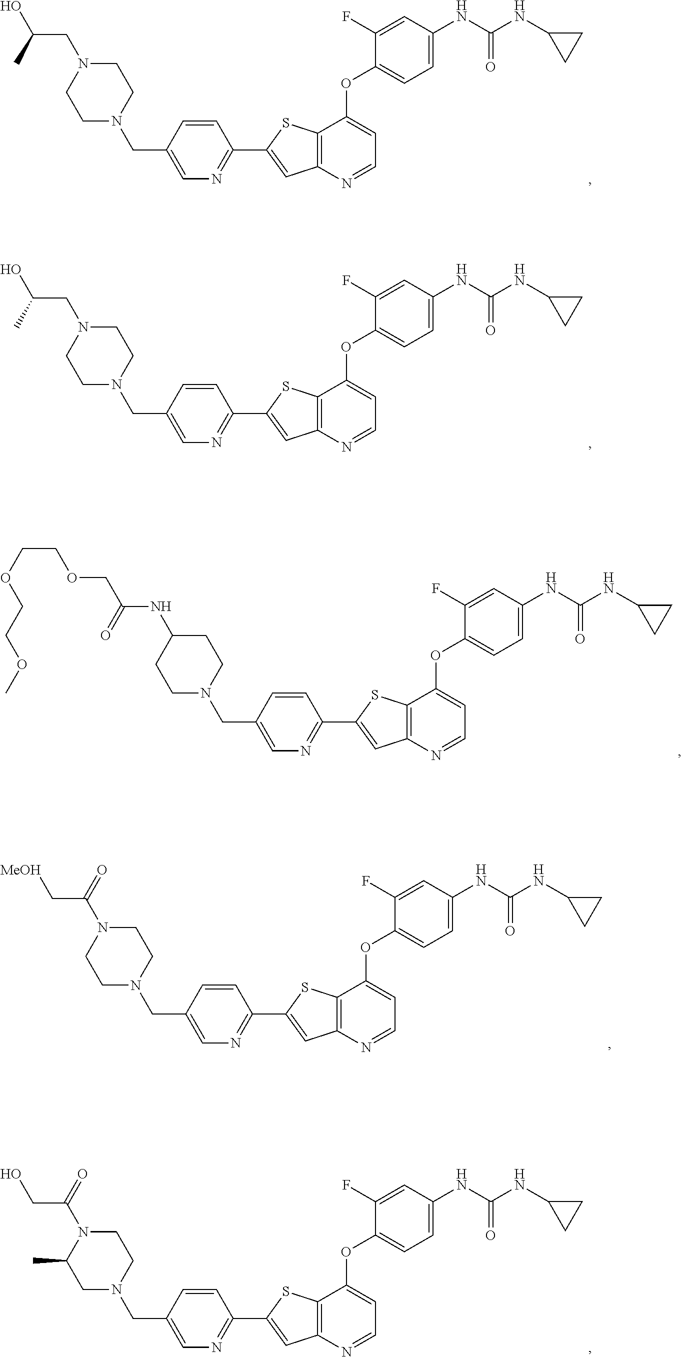 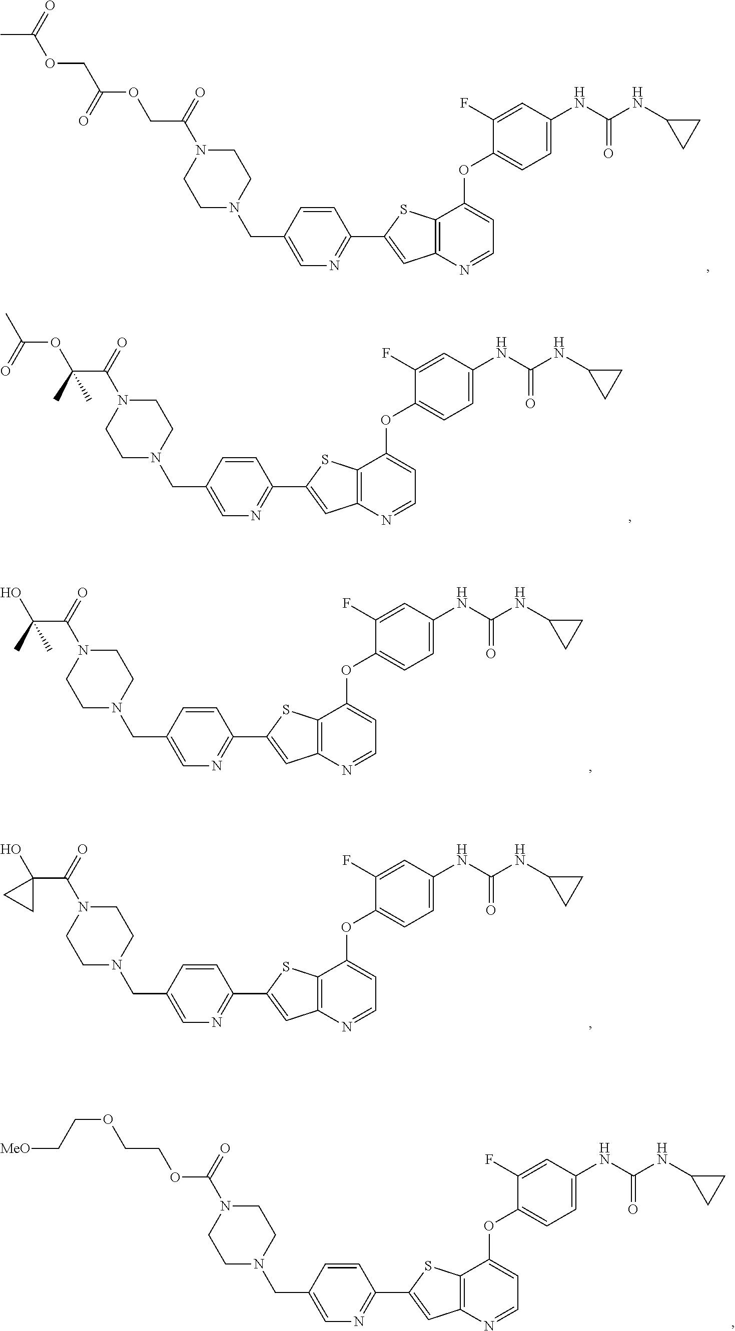 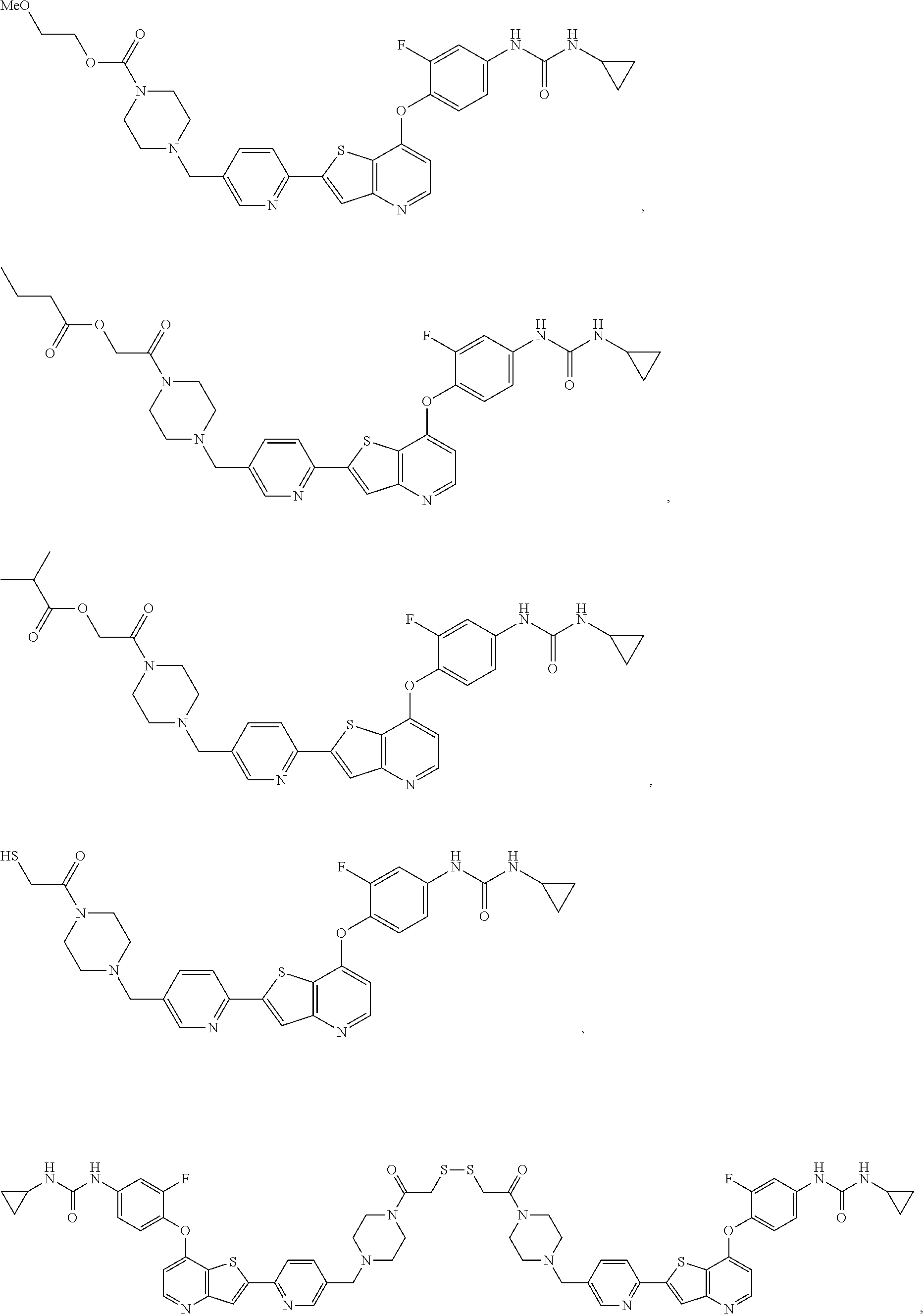 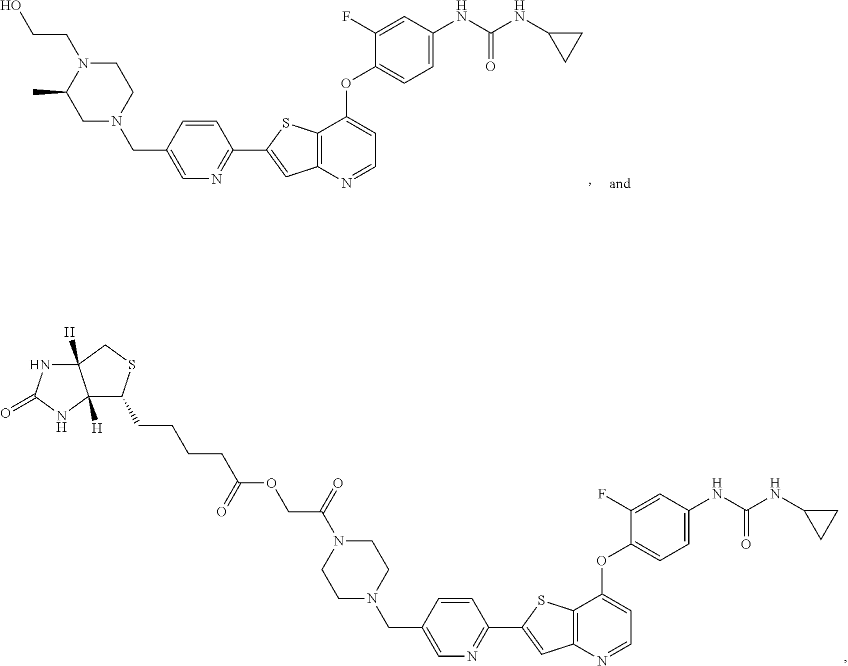 [SMILES: C[C@@H]1CN(Cc2ccc(-c3cc4nccc(Oc5ccc(NC(=O)NC6CC6)cc5F)c4s3)nc2)CCN1CCO ; "," ; "and" ; O=C(Nc1ccc(Oc2ccnc3cc(-c4ccc(CN5CCN(C(=O)COC(=O)CCCC[C@H]6SC[C@H]7NC(=O)N[C@H]76)CC5)cn4)sc23)c(F)c1)NC1CC1 ; ","]
and hydrates, solvates, pharmaceutically acceptable salts, prodrugs, soft drugs and complexes thereof, and racemic and scalemic mixtures, diastereomers and enantiomers thereof.

2. A composition comprising a compound according to claim 1 and a pharmaceutically acceptable carrier.

3. A method of treating an opthalmic disease, condition or disorder, the method comprising administering to a patient in need thereof a therapeutically effective amount of a compound according to claim 1 or a composition thereof, wherein the ophthalmic disease, disorder or condition is selected from the group consisting of (a) a disease, disorder or condition caused by choroidal angiogenesis, (b) diabetic retinopathy and (c) retinal oedema.

4. The method according to claim 3, wherein the ophthalmic disease, disorder or condition is age-related macular degeneration.
